(19) United States
(12) Patent Application Publication
(10) Pub. No.: US 2023/0391859 A1
(43) Pub. Date: Dec. 7, 2023

(54) ANTI-TRANSTHYRETIN ANTIBODIES AND METHODS OF USE THEREOF

(71) Applicant: Novo Nordisk A/S, Bagsvaerd (DK)

(72) Inventors: Frédérique Bard, Pacifica, CA (US); Gene Kinney, Boca Raton, FL (US); Wagner Zago, San Carlos, CA (US); Radhika Tripuraneni, Menlo Park, CA (US)

(21) Appl. No.: 18/033,568

(22) PCT Filed: Oct. 28, 2021
(86) PCT No.: PCT/US21/57150

§ 371 Date: Apr. 25, 2023

Related U.S. Application Data
(60) Provisional application No. 63/106,853, filed on Oct. 28, 2020.
(60) Provisional application No. 63/106,855, filed on Oct. 28, 2020.
(60) Provisional application No. 63/122,720, filed on Dec. 8, 2020.

(51) Int. Cl. C07K 16/18; A61P 25/02; A61P 9/00

(52) U.S. Cl. CPC C07K 16/18; A61P 25/02; A61P 9/00; A61K 2039/505

(57) ABSTRACT

Provided herein are methods and compositions for detecting, reducing, and inhibiting misfolded transthyretin protein.

### CROSS-REFERENCE TO RELATED APPLICATIONS

This application claims priority to U.S. Provisional Patent Application No. 63/106,853, filed on Oct. 28, 2020, U.S. Provisional Patent Application No. 63/106,855, filed on Oct. 28, 2020, and U.S. Provisional Patent Application No. 63/122,720, filed on Dec. 8, 2020. The contents of each application is incorporated by reference in its entirety.

### TECHNICAL FIELD

This document describes anti-transthyretin antibodies and methods of use thereof.

### BACKGROUND

Transthyretin (TTR) is a 127-amino acid, 55 kDa serum and cerebrospinal fluid transport protein primarily synthesized by the liver. In its native state, TTR exists as a tetramer and transports holo-retinol binding protein. In homozygotes, the tetramers comprise identical 127-amino-acid beta-sheet-rich subunits. In heterozygotes, the TTR tetramers are made up of variant and/or wild-type subunits, typically combined in a statistical fashion.

TTR can undergo extracellular misfolding and/or misassembly (amyloidogenesis) into a spectrum of aggregate structures is thought to cause degenerative diseases referred to as amyloid diseases. TTR undergoes conformational changes in order to become amyloidogenic. Dissociation of the TTR tetramer and partial unfolding exposes stretches of largely uncharged hydrophobic residues in an extended conformation that efficiently misassemble into largely unstructured spherical aggregates.

Transthyretin amyloidosis (ATTR) is a systemic disorder characterized by pathogenic, misfolded TTR and the extracellular deposition of amyloid fibrils composed of TTR. ATTR is generally caused by destabilization of the native TTR tetramer form (due to environmental or genetic conditions), leading to dissociation, misfolding, and aggregation of TTR into amyloid fibrils that accumulate in various organs and tissues, causing progressive dysfunction. See, e.g., Almeida and Saraiva, FEBS Letters 586:2891-2896 (2012); Ando et al., Orphanet Journal of Rare Diseases 8:31 (2013). Amyloid deposition is the cause of organ dysfunction and failure in both hereditary and wild type ATTR.

Conformation-specific monoclonal antibodies that detect misfolded TTR protein that bind non-native (e.g., misfolded) conformations of TTR, but not native TTR have been previously described. Such conformation-specific monoclonal antibodies may have therapeutic value to subjects with ATTR. For example, antibodies to misfolded-TTR have the potential to remove misfolded TTR, prevent deposition and enhance clearance of TTR amyloid in subjects and have been shown in vitro to inhibit TTR fibril formation and stimulate phagocytic uptake of aggregated TTR protein (Higaki, J., et al., Novel conformation-specific monoclonal antibodies against amyloidogenic forms of transthyretin, Amyloid, 23, 86-97 (2016), which is incorporated herein by reference in its entirety). Additionally, antibodies that detect misfolded TTR can be used to detect and quantitate misfolded-TTR proteins present in subjects.

Currently there are two therapeutic approaches that share a common principle—to slow TTR from entering the pathogenic pathway. This would, at least in theory, slow the formation of new amyloid. This is what therapies classified as stabilizers or silencers intend to do by either stabilizing the normal TTR tetramers or suppressing the production of the protein synthesis. However, simply preventing the protein from entering the pathogenic pathway is not adequate for patients who are at high risk of early mortality due to the substantial existing amyloid deposition in their vital organs. Instead, what is needed, is a mechanism that stops amyloid from causing harm by clearing it from tissue.

Presently, no therapies have been approved to clear amyloids (comprising misfolded TTR protein deposits) from organs and tissues. Thus, there remains a need for antibodies specific to misfolded TTR species suitable for administration to human subjects. Thus, disclosed herein are methods and compositions suitable for administration to subjects that decrease the amount of misfolded TTR that may clear misfolded TTR protein deposits—amyloids—from organs and tissues.

### SUMMARY

The present disclosure generally describes compositions and methods for inhibiting amyloid fibril formation, neutralizing soluble aggregate forms of misfolded TTR, and clearing insoluble amyloid fibrils through phagocytosis.

Provided herein are methods of reducing the level of misfolded transthyretin (misTTR) in a human subject including administering to a human subject in need thereof an amount of an antibody or an antigen-binding fragment thereof that reduces the level of misTTR, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of reducing the level of misfolded transthyretin (misTTR) in a human subject, the level of misTTR in the human subject is the plasma level of misTTR in the human subject.

In some embodiments of reducing the level of misfolded transthyretin (misTTR) in a human subject, the administering results in a reduction in the plasma level of misTTR in the human subject of about 10% to about 99%. In some embodiments of reducing the level of misfolded transthyretin (misTTR) in a human subject, the administering results in a reduction in the plasma level of misTTR in the human subject of about 20% to about 90%. In some embodiments of reducing the level of misfolded transthyretin (misTTR) in a human subject, the administering results in a reduction in the plasma level of misTTR in the human subject of about 40% to about 80%. In some embodiments of reducing the level of misfolded transthyretin (misTTR) in a human subject, the administering results in a reduction in the plasma level of misTTR in the human subject of about 50% to about 75%.

In some embodiments of reducing the level of misfolded transthyretin (misTTR) in a human subject, the antibody or the antigen-binding fragment thereof includes a heavy chain variable domain including SEQ ID NO: 7 and a light chain variable domain including SEQ ID NO: 8. In some embodiments of reducing the level of misfolded transthyretin (misTTR) in a human subject, the antibody or the antigen-binding fragment thereof includes a heavy chain including SEQ ID NO: 9 and a light chain including SEQ ID NO: 10.

Also provided herein are methods of opsonizing a transthyretin amyloid deposit in a human subject in need thereof including administering to a human subject in need thereof an amount of an antibody or an antigen-binding fragment thereof that opsonizes the transthyretin amyloid deposit, where the antibody or the antigen-binding fragment thereof includes a heavy chain variable domain including complementarily determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of methods of opsonizing a transthyretin amyloid deposit in a human subject in need thereof, the administering results in opsonization of about 50% to about 99% of transthyretin amyloid deposit(s) in the subject. methods of opsonizing a transthyretin amyloid deposit in a human subject in need thereof, the administering results in opsonization of about 80% to about 99% of transthyretin amyloid deposit(s) in the subject. methods of opsonizing a transthyretin amyloid deposit in a human subject in need thereof, the administering results in opsonization of about 90% to about 99% of transthyretin amyloid deposit(s) in the subject.

Also provided herein are methods of reducing the level of transthyretin amyloid deposits in a human subject in need thereof including administering to a human subject in need thereof an amount of an antibody or an antigen-binding fragment thereof that reduces the level of transthyretin amyloid deposits, where the antibody or the antigen-binding fragment thereof includes a heavy chain variable domain including complementarily determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of reducing the level of transthyretin amyloid deposits in a human subject in need thereof, the administering results in a reduction in the level of transthyretin amyloid deposits in the subject of about 50% to about 99%. In some embodiments of reducing the level of transthyretin amyloid deposits in a human subject in need thereof, the administering results in a reduction in the level of transthyretin amyloid deposits in the subject of about 80% to about 99%. In some embodiments of reducing the level of transthyretin amyloid deposits in a human subject in need thereof the administering results in a reduction in the level of transthyretin amyloid deposits in the subject of about 90% to about 99%.

In some embodiments of reducing the level of transthyretin amyloid deposits in a human subject in need thereof, the antibody or the antigen-binding fragment thereof includes a heavy chain variable domain including SEQ ID NO: 7 and a light chain variable domain including SEQ ID NO: 8. In some embodiments of reducing the level of transthyretin amyloid deposits in a human subject in need thereof, the antibody or the antigen-binding fragment thereof includes a heavy chain including SEQ ID NO: 9 and a light chain including SEQ ID NO: 10.

Also provided herein are methods of inhibiting transthyretin amyloid fibril formation in a human subject in need thereof including administering to a human subject in need thereof an amount of an antibody or an antigen-binding fragment thereof that inhibits transthyretin amyloid fibril formation, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of inhibiting transthyretin amyloid fibril formation in a human subject in need thereof, the administering results in about 50% to about 99% inhibition of transthyretin amyloid fibril formation in plasma of the human subject. In some embodiments of inhibiting transthyretin amyloid fibril formation in a human subject in need thereof, the administering results in about 80% to about 99% inhibition of transthyretin amyloid fibril formation in plasma of the human subject. In some embodiments of inhibiting transthyretin amyloid fibril formation in a human subject in need thereof, the administering results in about 90% to about 99% inhibition of transthyretin amyloid fibril formation in plasma of the human subject.

Also provided herein are methods of reducing the level of transthyretin amyloid fibrils in a human subject in need thereof including administering to a human subject in need thereof an amount of an antibody or an antigen-binding fragment thereof that reduces the level of transthyretin amyloid fibrils, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of reducing the level of transthyretin amyloid fibrils in a human subject in need thereof, the level of transthyretin amyloid fibrils is a plasma level of transthyretin amyloid fibrils.

In some embodiments of reducing the level of transthyretin amyloid fibrils in a human subject in need thereof, the administering results in a reduction of about 50% to about 99% in the plasma level of transthyretin amyloid fibrils in the human subject. In some embodiments of reducing the level of transthyretin amyloid fibrils in a human subject in need thereof, the administering results in a reduction of about 50% to about 99% in the plasma level of transthyretin amyloid fibrils in the human subject. In some embodiments of reducing the level of transthyretin amyloid fibrils in a human subject in need thereof, the administering results in a reduction of about 50% to about 99% in the plasma level of transthyretin amyloid fibrils in the human subject.

In some embodiments of reducing the level of transthyretin amyloid fibrils in a human subject in need thereof, the antibody or the antigen-binding fragment thereof includes a heavy chain variable domain including SEQ ID NO: 7 and a light chain variable domain including SEQ ID NO: 8. In some embodiments of reducing the level of transthyretin amyloid fibrils in a human subject in need thereof, the antibody or the antigen-binding fragment thereof includes a heavy chain including SEQ ID NO: 9 and a light chain including SEQ ID NO: 10.

Also provided herein are methods of neutralizing soluble aggregate forms of misTTR in a human subject in need thereof including administering to a human subject in need thereof an amount of an antibody or an antigen-binding fragment thereof that neutralizes soluble aggregate forms of misTRR, where the antibody or the antigen-binding fragment thereof includes a heavy chain variable domain including complementarily determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments methods of neutralizing soluble aggregate forms of misTTR in a human subject in need thereof, the administering results in about 50% to about 99% neutralization of soluble aggregate forms of misTTR in plasma of the human subject. In some embodiments methods of neutralizing soluble aggregate forms of misTTR in a human subject in need thereof, the administering results in about 80% to about 99% neutralization of soluble aggregate forms of misTTR in plasma of the human subject. In some embodiments methods of neutralizing soluble aggregate forms of misTTR in a human subject in need thereof, the administering results in about 90% to about 99% neutralization of soluble aggregate forms of misTTR in plasma of the human subject.

Also provided herein methods of reducing the level of soluble aggregate forms of misTTR in a human subject in need thereof including administering to a human subject in need thereof an amount of an antibody or an antigen-binding fragment thereof that reduces the level of soluble aggregate forms of misTTR, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of reducing the level of soluble aggregate forms of misTTR in a human subject in need thereof the level of soluble aggregate forms of misTTR in the human subject is a plasma level of soluble aggregate forms of misTTR in the human subject.

In some embodiments of reducing the level of soluble aggregate forms of misTTR in a human subject in need thereof, the administering results in an about 50% to about 99% reduction in the plasma level of soluble aggregate forms of misTTR in the human subject.

In some embodiments of reducing the level of soluble aggregate forms of misTTR in a human subject in need thereof, the administering results in an about 80% to about 99% reduction in the plasma level of soluble aggregate forms of misTTR in the human subject. In some embodiments of reducing the level of soluble aggregate forms of misTTR in a human subject in need thereof, the administering results in an about 90% to about 99% reduction in the plasma level of soluble aggregate forms of misTTR in the human subject.

In some embodiments of reducing the level of soluble aggregate forms of misTTR in a human subject in need thereof, the antibody or the antigen-binding fragment thereof includes a heavy chain variable domain including SEQ ID NO: 7 and a light chain variable domain including SEQ ID NO: 8. In some embodiments of reducing the level of soluble aggregate forms of misTTR in a human subject in need thereof, the antibody or the antigen-binding fragment thereof includes a heavy chain including SEQ ID NO: 9 and a light chain including SEQ ID NO: 10.

Also provided herein are methods of increasing phagocytosis of insoluble transthyretin amyloid fibrils in a human subject in need thereof including administering to a human subject in need thereof an amount of an antibody or an antigen-binding fragment thereof that increases phagocytosis of insoluble transthyretin amyloid fibrils, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of increasing phagocytosis of insoluble transthyretin amyloid fibrils in a human subject in need thereof, the administering results in a reduction in the level of insoluble transthyretin amyloid fibrils in the human subject. In some embodiments of increasing phagocytosis of insoluble transthyretin amyloid fibrils in a human subject in need thereof, the level of insoluble transthyretin amyloid fibrils in the subject is a plasma level of insoluble transthyretin amyloid fibrils in the human subject.

In some embodiments of increasing phagocytosis of insoluble transthyretin amyloid fibrils in a human subject in need thereof, the administering results in an about 50% to about 99% reduction in the plasma level of insoluble transthyretin amyloid fibrils in the subject. In some embodiments of increasing phagocytosis of insoluble transthyretin amyloid fibrils in a human subject in need thereof, the administering results in an about 80% to about 99% reduction in the plasma level of insoluble transthyretin amyloid fibrils in the subject. In some embodiments of increasing phagocytosis of insoluble transthyretin amyloid fibrils in a human subject in need thereof, the administering results in an about 90% to about 99% reduction in the plasma level of insoluble transthyretin amyloid fibrils in the subject.

Also provided herein are methods of decreasing the level of insoluble transthyretin amyloid fibrils in a human subject in need thereof including administering to a human subject in need thereof an amount of an antibody or an antigen-binding fragment thereof that decreases the level of insoluble transthyretin amyloid fibrils, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of decreasing the level of insoluble transthyretin amyloid fibrils in a human subject in need thereof, the level of insoluble transthyretin amyloid fibrils in the subject is a plasma level of insoluble transthyretin amyloid fibrils in the human subject.

In some embodiments of decreasing the level of insoluble transthyretin amyloid fibrils in a human subject in need thereof, the administering results in an about 50% to about 99% reduction in the plasma level of insoluble transthyretin amyloid fibrils in the subject. In some embodiments of decreasing the level of insoluble transthyretin amyloid fibrils in a human subject in need thereof, the administering results in an about 80% to about 99% reduction in the plasma level of insoluble transthyretin amyloid fibrils in the subject. In some embodiments of decreasing the level of insoluble transthyretin amyloid fibrils in a human subject in need thereof, the administering results in an about 90% to about 99% reduction in the plasma level of insoluble transthyretin amyloid fibrils in the subject.

In some embodiments of decreasing the level of insoluble transthyretin amyloid fibrils in a human subject in need thereof, the antibody or the antigen-binding fragment thereof includes a heavy chain variable domain including SEQ ID NO: 7 and a light chain variable domain including SEQ ID NO: 8. In some embodiments of decreasing the level of insoluble transthyretin amyloid fibrils in a human subject in need thereof, the antibody or the antigen-binding fragment thereof includes a heavy chain including SEQ ID NO: 9 and a light chain including SEQ ID NO: 10.

Also provided herein are methods of inhibiting formation of transthyretin amyloid deposits in a human subject in need thereof including administering to a human subject in need thereof an amount of an antibody or an antigen-binding fragment thereof that inhibits formation of transthyretin amyloid deposits, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of inhibiting formation of transthyretin amyloid deposits in a human subject in need thereof, the administering results in about 50% to about 99% inhibition of transthyretin amyloid fibril formation in plasma of the human subject. In some embodiments of inhibiting formation of transthyretin amyloid deposits in a human subject in need thereof, the administering results in about 80% to about 99% inhibition of transthyretin amyloid fibril formation in plasma of the human subject. In some embodiments of inhibiting formation of transthyretin amyloid deposits in a human subject in need thereof, the administering results in about 90% to about 99% inhibition of transthyretin amyloid fibril formation in plasma of the human subject.

Also provided herein are methods of treating amyloid transthyretin amyloidosis in a human subject in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of treating amyloid transthyretin amyloidosis in a human subject in need thereof, the antibody or the antigen-binding fragment thereof includes a heavy chain variable domain including SEQ ID NO: 7 and a light chain variable domain including SEQ ID NO: 8. In some embodiments of treating amyloid transthyretin amyloidosis in a human subject in need thereof, the antigen-binding fragment thereof includes a heavy chain including SEQ ID NO: 9 and a light chain including SEQ ID NO: 10.

Also provided herein are methods of improving neuropathy in a human subject with ATTR Amyloidosis in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1 SEQ ID NO: 2, and SEQ ID NO: 3, and a light chain variable domain including CDRs of SEQ ID NO: 4, SEQ ID NO: 5, and SEQ ID NO: 6.

In some embodiments of improving neuropathy in a human subject with ATTR amyloidosis in need thereof, administering results in a reduction of the neuropathy impairment score (NIS). In some embodiments of improving neuropathy in a human subject with ATTR amyloidosis in need thereof, the administering results in a reduced NIS of about 1 to about 15 in the human subject. In some embodiments of improving neuropathy in a human subject with ATTR amyloidosis in need thereof, administering results in a reduced NIS of about 5 to about 15 in the human subject. In some embodiments of improving neuropathy in a human subject with ATTR amyloidosis in need thereof, administering results in a reduced NIS of about 10 to about 15 in the human subject.

In some embodiments of improving neuropathy in a human subject with ATTR amyloidosis in need thereof, administering results in a reduction in NIS of at least about 1 in the human subject. In some embodiments of improving neuropathy in a human subject with ATTR amyloidosis in need thereof, administering results in a reduction in NIS of at least about 5 in the human subject. In some embodiments of improving neuropathy in a human subject with ATTR amyloidosis in need thereof, administering results in a reduction in NIS of at least about 10 in the human subject. In some embodiments of improving neuropathy in a human subject with ATTR amyloidosis in need thereof, administering results in a reduction in NIS of at least about 20 in the human subject.

Also provided herein are methods of treating ATTR amyloidosis in a human subject in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6, where the administering reduces the NIS in the human subject in need thereof.

In some embodiments of methods of treating ATTR amyloidosis in a human subject in need thereof, administering results in a reduced NIS of about 1 to about 15 in the human subject. In some embodiments of methods of treating ATTR amyloidosis in a human subject in need thereof, administering results in a reduced NIS of about 5 to about 15 in the human subject. In some embodiments of methods of treating ATTR amyloidosis in a human subject in need thereof, administering results in a reduced NIS of about 10 to about 15 in the human subject.

In some embodiments of methods of treating ATTR amyloidosis in a human subject in need thereof, administering results in a reduction in NIS of at least about 1 in the human subject. In some embodiments of methods of treating ATTR amyloidosis in a human subject in need thereof, administering results in a reduction in NIS of at least about 5 in the human subject. In some embodiments of methods of treating ATTR amyloidosis in a human subject in need thereof, administering results in a reduction in NIS of at least about 10 in the human subject. In some embodiments of methods of treating ATTR amyloidosis in a human subject in need thereof, administering results in a reduction in NIS of at least about 20 in the human subject.

Also provided herein are methods of improving cardiac systolic function in a human subject with ATTR Amyloidosis in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6.

In some embodiments of methods of improving cardiac systolic function in a human subject with ATTR Amyloidosis in need thereof, administering results in a reduction of global longitudinal strain (GLS). In some embodiments of methods of improving cardiac systolic function in a human subject with ATTR Amyloidosis in need thereof, administering results in a reduction of GLS of about 0.1% to about 10% in the human subject. In some embodiments of methods of improving cardiac systolic function in a human subject with ATTR Amyloidosis in need thereof, administering results in a reduction of GLS of about 0.5% to about 10% in the human subject. In some embodiments of methods of improving cardiac systolic function in a human subject with ATTR Amyloidosis in need thereof, administering results in a reduction of GLS of about 1% to about 10% in the human subject.

In some embodiments of methods of improving cardiac systolic function in a human subject with ATTR Amyloidosis in need thereof, administering results in a reduction of GLS of at least about 0.5% in the human subject.

In some embodiments of methods of improving cardiac systolic function in a human subject with ATTR Amyloidosis in need thereof, administering results in a reduction of GLS of at least about 1.0% in the human subject. In some embodiments of methods of improving cardiac systolic function in a human subject with ATTR Amyloidosis in need thereof, administering results in a reduction of GLS of at least about 2.0% in the human subject. In some embodiments of methods of improving cardiac systolic function in a human subject with ATTR Amyloidosis in need thereof, administering results in a reduction of GLS of at least about 10% in the human subject.

Also provided herein are methods of treating ATTR amyloidosis in a human subject in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6, where the administering reduces the global longitudinal strain (GLS) in the human subject in need thereof.

In some embodiments of methods of treating ATTR amyloidosis in a human subject in need thereof including administering to a human subject in need thereof, administering results in a reduction of GLS of about 0.1% to about 10% in the human subject. In some embodiments of methods of treating ATTR amyloidosis in a human subject in need thereof including administering to a human subject in need thereof, administering results in a reduction of GLS of about 0.5% to about 10% in the human subject. In some embodiments of methods of treating ATTR amyloidosis in a human subject in need thereof including administering to a human subject in need thereof, administering results in a reduction of GLS of about 1% to about 10% in the human subject.

In some embodiments of methods of treating ATTR amyloidosis in a human subject in need thereof including administering to a human subject in need thereof, administering results in a reduction of GLS of at least about 0.5% in the human subject. In some embodiments of methods of treating ATTR amyloidosis in a human subject in need thereof including administering to a human subject in need thereof, administering results in a reduction of GLS of at least about 1.0% in the human subject. In some embodiments of methods of treating ATTR amyloidosis in a human subject in need thereof including administering to a human subject in need thereof, administering results in a reduction of GLS of at least about 2.0% in the human subject. In some embodiments of methods of treating ATTR amyloidosis in a human subject in need thereof including administering to a human subject in need thereof, administering results in a reduction of GLS of at least about 10% in the human subject.

In some embodiments, the antibody or the antigen-binding fragment thereof includes a heavy chain variable domain including SEQ ID NO: 7 and a light chain variable domain including SEQ ID NO: 8. In some embodiments, the antibody or the antigen-binding fragment thereof includes a heavy chain including SEQ ID NO: 9 and a light chain including SEQ ID NO: 10.

Also provided herein are methods of treating heart failure in a human subject with ATTR Amyloidosis in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6, where the New York Heart Association (NYHA) class of the human subject in need thereof does not increase.

In some embodiments, administering does not result in an increase in the NYHA class after a period of time sufficient to treat NYHA class I, II, or III in the human subject.

Also provided herein are methods of treating heart failure in a human subject with ATTR Amyloidosis in need thereof including administering to a human subject in need thereof a therapeutically effective amount of an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6, where the administering results in an improved NYHA class after a period of time sufficient to treat NYHA class I, II, III, or IV in the human subject.

In some embodiments of any of the methods described herein, the human subject has been previously diagnosed or identified as having amyloid transthyretin amyloidosis. In some embodiments of any of the methods described herein, the amyloid transthyretin amyloidosis is hereditary amyloid transthyretin amyloidosis. In some embodiments of any of the methods described herein, the amyloid transthyretin amyloidosis is wild-type amyloidosis.

In some embodiments of the methods herein, administering includes administering between about 0.1 mg/kg to about 40 mg/kg of the antibody or the antigen-binding antibody fragment thereof. In some embodiments of the methods herein, administering includes administering between about 1 mg/kg to about 30 mg/kg of the antibody or the antigen-binding antibody fragment thereof. In some embodiments of the methods herein, administering includes administering between about 3 mg/kg to about 10 mg/kg or about 10 mg/kg to about 30 mg/kg of the antibody or the antigen-binding antibody fragment thereof. In some embodiments of the methods herein, administering includes administering about 0.1 mg/kg, about 0.3 mg/kg, about 1 mg/kg, about 3 mg/kg, about 10 mg/kg, or about 30 mg/kg of the antibody or the antigen-binding antibody fragment thereof.

In some embodiments of the methods herein, administering includes intravenous administration.

In some embodiments of the methods herein, the administering is performed at an interval of about every 14 days, about every 2 weeks, about every 3 weeks, about every 28 days, about every 4 weeks, about monthly, about every 5 weeks, about every 6 weeks, about every 7 weeks, about every 8 weeks, or about every 2 months. In some embodiments of the methods herein, administering is performed over a period of time of about 1 month to about 1 year.

Also provided herein are kits including an antibody or an antigen-binding fragment thereof, where the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain including CDRs of SEQ ID NO: 4, SEQ ID NO: 5, and SEQ ID NO: 6; and instructions for performing any of the methods described herein.

In some kits including an antibody or an antigen-binding fragment thereof, the antibody or the antigen-binding fragment thereof includes a heavy chain variable domain including SEQ ID NO: 7 and a light chain variable domain including SEQ ID NO: 8.

In some kits including an antibody or an antigen-binding fragment thereof, the antibody or the antigen-binding fragment thereof includes a heavy chain including SEQ ID NO: 9 and a light chain including SEQ ID NO: 10.

All publications, patents, patent applications, and information available on the internet and mentioned in this specification are herein incorporated by reference to the same extent as if each individual publication, patent, patent application, or item of information was specifically and individually indicated to be incorporated by reference

To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions will be evident to those skilled in the art without departing from the scope of this disclosure. It should also be understood that various alternatives to the specific embodiments described herein are also within the scope of this disclosure.

### DESCRIPTION OF DRAWINGS

FIGS. 1A-B are graphs showing the concentration (μg/ml) of PRX004 over time after the first dose in month 1 (FIG. 1A) and after the dose in month 3 (FIG. 1B) for cohorts 1-6.

FIGS. 2A-C are graphs showing ATTR amyloid binding for PRX004 doses.

FIGS. 3A-B are graphs showing the amount of misTTR as the percent of baseline over time after administration of PRX004 in month 1 (FIG. 3A) and after the dose administered in month 3 (FIG. 3B) for cohorts 3, 4, 5, and 6.

FIG. 4A is graph showing the dose-dependent decrease in misTTR for cohorts 3, 4, 5, and 6. FIG. 4B is a graph showing total transthyretin protein (% baseline) over time after a dose of PRX004. FIG. 4C is a graph showing transthyretin protein (% baseline) over time after a dose of PRX004.

FIGS. 5A-G are images showing binding of a control antibody (FIG. 5A) and various concentrations of the murine precursor of PRX004 (mPRX004) (FIGS. 5B-F) to cardiac amyloid deposits. FIG. 5G is a graph showing a binding curve of mPRX004 to cardiac amyloid deposits. FIGS. 5H-M are images showing binding of a control antibody (FIG. 5H) and various concentrations of mPRX004 in cardiac control tissue (FIGS. 5I-M).

FIG. 6A is a graph showing changes in neuropathy impairment score (NIS) from baseline over a treatment period. FIG. 6B is a graph showing changes in NIS from baseline over a treatment period.

FIG. 7 is a graph showing changes in global longitudinal strain (GLS) from baseline over a treatment period. Dots with arrows are patients who received PRX004 alone.

FIG. 8 is a graph showing inhibition of fibril formation.

FIGS. 9A-D are images showing binding of mPRX004 to cardiac tissue (FIGS. 9A-B), sciatic nerve tissue (FIG. 9C), and GI tract tissue (FIG. 9D).

FIG. 10 is a graph showing percentage of cells with internalized ATTR.

### DETAILED DESCRIPTION

To facilitate understanding of the disclosure set forth herein, terms are defined below. Generally, the nomenclature used herein and the laboratory procedures in biochemistry, genetics, molecular biology, and molecular diagnostics described herein are those well-known and commonly employed in the art. Other features, objects, and advantages of the invention will be apparent from the description and drawings, and from the claims. Unless defined otherwise, all technical and scientific terms used herein generally have the same meaning as commonly understood by one of ordinary skill in the art to which this disclosure belongs. Methods and materials are described herein for use in the present invention; other, suitable methods and materials known in the art can also be used. The materials, methods, and examples are illustrative only and not intended to be limiting. Each of the patents, applications, published applications, and other publications that are mentioned throughout the specification and the attached appendices are incorporated herein by reference in their entireties. In case of conflict, the present specification, including definitions, will control.

Monoclonal antibodies or other biological entities are typically provided in isolated form. This means that an antibody or other biologically entity is typically at least 50% w/w pure of interfering proteins and other contaminants arising from its production or purification but does not exclude the possibility that the monoclonal antibody is combined with an excess of pharmaceutically acceptable carrier(s) or other vehicle intended to facilitate its use. Sometimes monoclonal antibodies are at least 60%, 70%, 80%, 90%, 95% or 99% w/w pure of interfering proteins and contaminants from production or purification. Often an isolated monoclonal antibody or other biological entity is the predominant macromolecular species remaining after its purification.

Specific binding of an antibody to its target antigen means an affinity of at least 106, 107, 108, 109, or 1010 M−1, Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology, Paul, W., ed., 2nd ed. Raven Press, N.Y., 1989, Ch. 7 (incorporated by reference in its entirety for all purposes).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, Md., 1987 and 1991), The Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al (bioinfo.org.uk/abs) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

The term “antibody” includes intact antibodies and binding fragments thereof. Typically, fragments compete with the intact antibody from which they were derived for specific binding to the target including separate heavy chains, light chains Fab, Fab′, F(ab′)2, F(ab)c. Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)). In some bispecific antibodies, the two different heavy/light chain pairs include a humanized PRX004 heavy chain/light chain pair and a heavy chain/light chain pair specific for a different epitope on transthyretin than that bound by PRX004.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “native” with respect to the structure transthyretin (TTR) refers to the normal folded structure of TTR in its properly functioning state (i.e., a TTR tetramer). As TTR is a tetramer in its natively folded form, non-native forms of TTR include, for example, misfolded TTR tetramers, TTR monomers, aggregated forms of TTR, and fibril forms of TTR. Non-native forms of TTR can include molecules comprising wild-type TTR amino acid sequences or mutations.

The term “misfolded” with respect to TTR refers to the secondary and tertiary structure of a TTR polypeptide monomer or multimer, and indicates that the polypeptide has adopted a conformation that is not normal for that protein in its properly functioning state. Although TTR misfolding can be caused by mutations in the protein (e.g., deletion, substitution, or addition), wild-type TTR proteins can also be misfolded in diseases, exposing specific epitopes.

The term “pharmaceutically acceptable” means that the carrier, diluent, excipient, or auxiliary is compatible with the other ingredients of the formulation and not substantially deleterious to the recipient thereof.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “control sample” refers to a biological sample not known or suspected to include monomeric, misfolded, aggregated, or fibril forms of transthyretin (TTR), such as in TTR amyloid deposits. Control samples can be obtained from individuals not afflicted with a TTR amyloidosis or a specifically chosen type of TTR amyloidosis. Alternatively, control samples can be obtained from patients afflicted with TTR amyloidosis or a specifically chosen type of TTR amyloidosis. Such samples can be obtained at the same time as a biological sample thought to comprise the TTR amyloidosis or on a different occasion. A biological sample and a control sample can both be obtained from the same tissue (e.g., a tissue section containing both TTR amyloid deposits and surrounding normal tissue). Preferably, control samples consist essentially or entirely of tissue free of TTR amyloid deposits and can be used in comparison to a biological sample thought to comprise TTR amyloid deposits. Preferably, the tissue in the control sample is the same type as the tissue in the biological sample (e.g., cardiomyocytes in the heart). In some embodiments, a control sample or level can be a level in the same subject prior to administration of any of the antibodies or antigen-binding fragments thereof described herein.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “treat” or “treating” refers to a reduction in one or more of the severity, frequency, duration, or number of signs or symptoms of a disease observed in a subject.

Designation of a range of values includes all integers within or defining the range, and all subranges defined by integers within the range.

Unless otherwise apparent from the context, the term “about” encompasses values within a standard margin of error of measurement (e.g., SEM) of a stated value.

Statistical significance means p≤0.05.

The term “symptom” refers to a subjective evidence of a disease, such as altered gait, as perceivable by a subject. A “sign” refers to objective evidence of a disease as observable by a physician.

For purposes of classifying amino acids substitutions as conservative or nonconservative, amino acids are grouped as follows: Group I (hydrophobic side chains): met, ala, val, leu, ile; Group II (neutral hydrophilic side chains): asn, gln, cys, ser, thr; Group III (acidic side chains): asp, glu; Group IV (basic side chains): his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” or “including” one or more recited elements may include other elements not specifically recited. For example, a composition that “comprises” or “includes” an antibody may contain the antibody alone or in combination with other ingredients.

Transthyretin amyloidosis (ATTR) is a rare, progressive, and often fatal disease characterized by the deposition of misfolded transthyretin protein primarily in the heart and peripheral nerves, which causes significant morbidity and mortality. These protein deposits most frequently occur in the peripheral nervous system, which is composed of nerves connecting the brain and spinal cord to muscles and sensory cells that detect sensations such as touch, pain, heat, and sound. Protein deposits in these nerves result in a loss of sensation in the extremities (peripheral neuropathy). Other systems, including the autonomic nervous system, which controls involuntary body functions such as blood pressure, heart rate, and digestion, can also be affected by amyloidosis. In some cases, the brain and spinal cord (central nervous system) are affected. Other areas of amyloidosis include the heart, kidneys, eyes, and gastrointestinal tract. There are three types of ATTR amyloidosis: hereditary ATTR with cardiomyopathy (hATTR-CM) (formerly known as FAC); hereditary ATTR with polyneuropathy (hATTR-PN) (formerly known as FAP); and wild-type ATTR (wtATTR). hATTR-CM and hATTR-PN are hereditary, whereas wtATTR is not hereditary and occurs sporadically.

Specifically, ATTR is caused by the deposition of misfolded transthyretin (TTR) protein in various tissues of the body, which can lead to organ dysfunction. Conformation-specific monoclonal antibodies that detect misfolded TTR protein that bind non-native (e.g., misfolded) conformations of TTR, but not native TTR have been previously described. Such conformation-specific monoclonal antibodies may have therapeutic value to subjects with ATTR. For example, antibodies to misfolded-TTR have the potential to prevent deposition and enhance clearance of TTR amyloid in subjects with ATTR amyloidosis and have been shown in vitro to inhibit TTR fibril formation and stimulate phagocytic uptake of aggregated TTR protein (Higaki, J., et al., Novel conformation-specific monoclonal antibodies against amyloidogenic forms of transthyretin, Amyloid, 23, 86-97 (2016), which is incorporated herein by reference in its entirety). Additionally, antibodies that detect misfolded can be used to detect and quantitate misfolded-TTR proteins present in ATTR subjects.

Presently, no therapies have been approved to clear misfolded-TTR protein deposits from organs and tissues.

Transthyretin (TTR) is a 127-amino acid, 55 kDa serum and cerebrospinal fluid transport protein primarily synthesized by the liver. It has also been referred to as prealbumin, thyroxine binding prealbumin, ATTR, and TBPA. In its native state, TTR exists as a tetramer. In homozygotes, the tetramers comprise identical 127-amino-acid beta-sheet-rich subunits. In heterozygotes, the TTR tetramers are made up of variant and/or wild-type subunits, typically combined in a statistical fashion.

The established function of TTR in the blood is to transport holo-retinol binding protein. Although TTR is the major carrier of thyroxine (T4) in the blood of rodents, utilizing binding sites that are orthogonal to those used for holo-retinol binding protein, the T4 binding sites are effectively unoccupied in humans.

TTR is one of at least thirty different human proteins whose extracellular misfolding and/or misassembly (amyloidogenesis) into a spectrum of aggregate structures is thought to cause degenerative diseases referred to as amyloid diseases. TTR undergoes conformational changes in order to become amyloidogenic. Dissociation of the TTR tetramer and partial unfolding exposes stretches of largely uncharged hydrophobic residues in an extended conformation that efficiently misassemble into largely unstructured spherical aggregates that ultimately undergo conformation conversion into cross-beta sheet amyloid structures.

Transthyretin (TTR) amyloidosis is a systemic disorder characterized by pathogenic, misfolded TTR and the extracellular deposition of amyloid fibrils composed of TTR. TTR amyloidosis is generally caused by destabilization of the native TTR tetramer form (due to environmental or genetic conditions), leading to dissociation, misfolding, and aggregation of TTR into amyloid fibrils that accumulate in various organs and tissues, causing progressive dysfunction. See, e.g., Almeida and Saraiva, FEBS Letters 586:2891-2896 (2012); Ando et al., Orphanet Journal of Rare Diseases 8:31 (2013).

In humans, both wild-type TTR tetramers and mixed tetramers comprised of mutant and wild-type subunits can dissociate, misfold, and aggregate, with the process of amyloidogenesis leading to the degeneration of affected tissue. Thus, TTR amyloidoses encompass diseases caused by pathogenic misfolded TTR resulting from mutations in TTR or resulting from non-mutated, misfolded TTR.

For example, wild-type ATTR amyloidosis (also called senile systemic amyloidosis or SSA) and senile cardiac amyloidosis (SCA) are age-related types of amyloidosis that result from the deposition of wild-type TTR amyloid outside and within the cardiomyocytes of the heart. TTR amyloidosis is also the most common form of hereditary (familial) amyloidosis, which is caused by mutations that destabilize the TTR protein. The TTR amyloidoses associated with point mutations in the TTR gene include familial amyloid polyneuropathy (FAP), familial amyloid cardiomyopathy (FAC), and the rare central nervous system selective amyloidosis (CNSA). Patients with hereditary (familial) TTR amyloidosis are almost always heterozygotes, meaning that the TTR tetramers are composed of mutant and/or wild-type TTR subunits, generally statistically distributed. Hereditary (familial) versions of TTR amyloidosis are generally autosomal dominant and are typically earlier onset than the sporadic diseases (SSA and SCA).

There are over 100 mutations in the gene encoding TTR that have been implicated in the autosomal dominant disorders FAP and FAC. See, e.g., US 2014/0056904; Saraiva, Hum. Mutat. 17(6):493-503 (2001); Damas and Saraiva, J. Struct. Biol. 130:290-299; Dwulet and Benson, Biochem. Biophys. Res. Commun. 114:657-662 (1983). These amyloid-causing mutations are distributed throughout the entire molecule of TTR. Generally, the more destabilizing the mutant subunits are to the TTR tetramer structure, the earlier the onset of amyloid disease. The pathogenic potential of a TTR variant is generally determined by a combination of its instability and its cellular secretion efficiency. The initial pathology caused by some TTR variants comes from their selective destruction of cardiac tissue, whereas that from other TTR variants comes from compromising the peripheral and autonomic nervous system. The tissue damage caused by TTR amyloidogenesis appear to stem largely from the toxicity of small, diffusible TTR aggregates, although accumulation of extracellular amyloid may contribute and almost certainly compromises organ structure in the late stages of the TTR amyloidosis. Exemplary TTR mutations include V30M, Y114C, G47R, 5501, E61L, T49S, F33V, A45T, E89K, E89Q, and V122I.

TTR amyloidosis presents in many different forms, with considerable phenotypic variation across individuals and geographic locations. For example, TTR amyloidosis can present as a progressive, axonal sensory autonomic and motor neuropathy. TTR amyloidosis can also present as an infiltrative cardiomyopathy.

The age at onset of disease-related symptoms varies between the second and ninth decades of life, with great variations across different populations. The multisystem involvement of TTR amyloidosis is a clue to its diagnosis. For example, TTR amyloidosis diagnosis is considered when one or several of the following are present: (1) family history of neuropathic disease, especially associated with heart failure; (2) neuropathic pain or progressive sensory disturbances of unknown etiology; (3) carpal tunnel syndrome without obvious cause, particularly if it is bilateral and requires surgical release; (4) gastrointestinal motility disturbances or autonomic nerve dysfunction of unknown etiology (e.g., erectile dysfunction, orthostatic hypotension, neurogenic bladder); (5) cardiac disease characterized by thickened ventricular walls in the absence of hypertension; (6) advanced atrio-ventricular block of unknown origin, particularly when accompanied by a thickened heart; and (6) vitreous body inclusions of the cotton-wool type. See Ando et al., Orphanet Journal of Rare Diseases 8:31 (2013). Other symptoms can include, for example, polyneuropathy, sensory loss, pain, weakness in lower limbs, dyshidrosis, diarrhea, constipation, weight loss, and urinary incontinence/retention.

Diagnosis of TTR amyloidosis typically relies on target organ biopsies, followed by histological staining of the excised tissue with the amyloid-specific dye, Congo red. If a positive test for amyloid is observed, immunohistochemical staining and mass spectroscopic identification of TTR is subsequently performed to ensure that the precursor protein responsible for amyloid formation is indeed TTR. Antibodies disclosed herein are useful in distinguishing TTR amyloidosis from a non-TTR amyloidosis e.g. amyloid light-chain (AL) amyloidosis, also known as primary systemic amyloidosis, For familial forms of the diseases, demonstration of a mutation in the gene encoding TTR is then needed before diagnosis can be made. This can be accomplished, for example; through isoelectric focusing electrophoresis, polymerase chain reaction, or laser dissection/liquid chromatography-tandem mass spectrometry. See, e.g., US 2014/0056904; Ruberg and Berk, Circulation 126:1286-1300 (2012); Ando et al., Orphanet Journal of Rare Diseases 8:31 (2013).

Antibodies of the invention can be administered concomitant with another treatment for the same indication as the antibody, meaning that the other treatment is administered at least once during the period in which the antibody is administered, such period beginning one month before the first dosing and ending one month after the last dosing of the antibody. The other treatment can be administered at recurring intervals during this period, which may or may not be the same as the intervals at which the antibody is administered. The other treatment may be a symptomatic treatment.

PRX004 is a monoclonal antibody (mAb) that targets monomeric and misfolded TTR. PRX004 immunoreacts specifically to a cryptic epitope found in the misfolded ATTR amyloid deposits in various organs and misfolded monomers (amyloid precursors) from both wild-type ATTR (wtATTR) and hereditary ATTR (hATTR) amyloidosis patients, while not reacting with the normally folded TTR(e.g., tetramers). In preclinical studies, upon binding in vitro, PRX004 has been shown to inhibit amyloid fibril formation, neutralize soluble aggregated forms of misfolded TTR, and promote clearance of insoluble amyloid fibrils.

Currently available treatment options in the United States (US) for hATTR amyloidosis include liver transplant and supportive care, depending on the organ(s) involved, patisiran and inotersen for treatment of the polyneuropathy of hATTR in adults, and tafamidis for treatment of the cardiomyopathy of wtATTR and hATTR in adults. The same treatment options are available in Europe, with the exception of tafamidis for the treatment of cardiomyopathy, the addition of tafamidis for patients with Stage 1 peripheral neuropathy, and inotersen and patisiran for the treatment of Stage 1 or 2 polyneuropathy in adult patients with hATTR amyloidosis. While published data from tetramer stabilizers suggest a possible slowing of disease progression (Waddington, 2016), a high unmet medical need still exists as these therapies do not target the removal of amyloid already deposited in tissues. Without wishing to be bound by any theory, in one proposed mechanism of action, PRX004 may remove amyloid by binding to and opsonizing the ATTR deposits, and engaging tissue resident macrophages to clear the deposits via phagocytosis which may improve organ function. We call this a depleter mechanism of action which is different from the TTR stabilizers and silencers currently prescribed for ATTR. If misfolded soluble TTR species are also toxic to nerves or cardiomyocytes, PRX004 may bind and neutralize these toxic species as well. Thus, PRX004 has the potential to demonstrate significant improvements in efficacy compared with current standard of care.

PRX004 is a humanized immunoglobulin (Ig) G1 kappa mAb that specifically binds a unique epitope, amino acid residues 89-97 (SEQ ID NO: 11 EHAEVVFTA) of TTR, that is exposed only on monomeric, misfolded, and aggregated forms of TTR but hidden in the native tetramer conformation. This epitope is situated within an aggregation-prone region of the β-strand F of the TTR protein recently found to be directly involved in the TTR aggregation process (Saelices, L., et al., Uncovering the mechanism of aggregation of human transthyretin, J Biol Chem. 290(48): 28932-43. 2015, which is incorporated herein by reference in its entirety) and is an important differentiating epitope between misfolded and native forms of the protein. Binding of PRX004 to this region impedes the self-association of pathogenic, misfolded monomers and oligomers required for fibril formation, thereby preventing TTR amyloid formation and deposition.

Supportive evidence for this mode of action comes from immunotherapy with antibodies that target other amyloidogenic proteins. Specifically, mAbs for Alzheimer's disease have demonstrated a great level of concordance between in vitro phagocytosis and efficacy in vivo (Bard, 2000; Sevigny, J., The antibody aducanaumab reduces Aβ plaques in Alzheimer's disease, Nature, 537(7618):50-6 (2016); each of which is incorporated herein by reference in its entirety). Furthermore, Bard, 2003 (Bard, F. et al., Epitope and isotype specificities of antibodies to beta-amyloid peptide for protection against Alzheimer's disease-like neuropathology. Proc Natl Acad Sci USA 100(4):2023-8 Epub 3 February (2003), which is incorporated herein by reference in its entirety) reported that only antibodies that induced phagocytosis in vivo were able to clear plaques from brains of Aβ plaque-bearing transgenic mice. In clinical studies, anti-Aβ antibodies were demonstrated to enter the central nervous system and induce clearance of pre-existing amyloid deposits from brains of Alzheimer's patients (Liu, E., et al., Amyloid-β 11C-PiB-PET imaging results from 2 randomized bapineuzumab phase 3 AD trials, Neurology, 85(8):692-700 doi:10.1212/WNL.0000000000001877 Epub 24 July (2015); Rinne, J. O., et al., 11C-PiB PET assessment of change in fibrillary amyloid-beta load in patients with Alzheimer's disease treated with bapineuzumab: a phase 2, double-blind, placebo-controlled, ascending-dose study, Lancet Neurol. 9(4):363-72 (2010); Sevigny, 2016, each of which is incorporated herein by reference in its entirety).

Disclosed herein is an antibody having (e.g., PRX004) some or all (e.g.; 3, 4, and 6) CDRs. The antibodies or antigen-binding fragments thereof described herein can include a heavy chain variable domain that has at least two, and usually all three CDRs including SEQ ID NO: 1, SEQ ID NO: 2, and SEQ ID NO: 3 corresponding to heavy chain CDR1, CDR2, and CDR3, respectively. The antibodies or antigen-binding fragments thereof described herein can include a light chain variable domain that has at least two and usually all three CDRs including SEQ ID NO: 4, SEQ ID NO: 5, and SEQ ID NO: 6 corresponding to light chain CDR1, CDR2, and CDR3, respectively. In some embodiments, the antibodies or antigen-binding fragments thereof described herein can include a heavy chain variable domain including CDRs 1-3 corresponding to SEQ ID NO: 1, SEQ ID NO: 2, and SEQ ID NO: 3, respectively, and a light chain variable domain including CDRs 1-3 corresponding to SEQ ID NO: 4, SEQ ID NO: 5, and SEQ ID NO: 6, respectively.

In some embodiments, the antibodies or antigen-binding fragments thereof described herein can include a heavy chain variable domain comprising SEQ ID NO: 7. In some embodiments, PRX004 can include a light chain variable domain comprising SEQ ID NO: 8. In some embodiments, the antibodies or antigen-binding fragments thereof described herein can include a heavy chain variable domain comprising SEQ ID NO: 7 and a light chain variable domain comprising SEQ ID NO: 8. In some embodiments, the antibodies or antigen-binding fragments thereof described herein can include a heavy chain comprising SEQ ID NO: 9. In some embodiments, the antibodies or antigen-binding fragments thereof described herein can include a light chain comprising SEQ ID NO: 10. In some embodiments, the antibodies or antigen-binding fragments thereof described herein can include a heavy chain including SEQ ID NO: 9 and a light chain including SEQ ID NO: 10. In some embodiment, the antibodies or antigen-binding fragments thereof described herein includes a heavy chain that does not comprise a C-terminal lysine. For example, in one such embodiment, the C-terminal lysine included in SEQ ID NO: 9 is absent.

The antibodies or antigen-binding fragments thereof described herein can inhibit or reduce aggregation of TTR, inhibit or reduce TTR fibril formation, reduce or clear TTR deposits or aggregated TTR, or stabilize non-toxic conformations of TTR in an animal model.

Also provided herein are methods for reducing the level of misfolded transthyretin (misTTR) in a human subject including administering to a human subject in need thereof an amount of an antibody or an antigen-binding fragment thereof that reduces the level of misTTR, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibodies or antigen-binding fragments thereof described herein reduces the level of misTTR in plasma by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to a level of misTTR in plasma in the human subject prior to administration). In some embodiments, administering the antibodies or antigen-binding fragments thereof described herein reduces the level of misTTR in plasma by about 10% to about 99%, about 20% to about 90%, about 30% to about 80%, about 40% to 80%, or about 50% to 75% (e.g., as compared to a level of misTTR in plasma in the human subject prior to administration). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to the level of misTTR in plasma in the human subject prior to the administering) (e.g., when administered using any of the exemplary dosages and/or frequencies described herein).

Also provided herein are methods of opsonizing a transthyretin amyloid deposit in a human subject in need thereof including administering to a human subject in need thereof an amount of an antibody or an antigen-binding fragment thereof that opsonizes the transthyretin amyloid deposit, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof opsonizes transthyretin amyloid deposits in plasma by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In some embodiments, administering the antibody or the antigen-binding fragment thereof opsonizes transthyretin amyloid deposits in plasma by about 10% to about 90%, about 20% to about 80%, about 30% to about 70%, about 40% to 60%, about 50% to about 99%, about 80% to about 99%, or about 90 to 99%. In some embodiments, the administering of the antibody or the antigen-binding fragment thereof opsonizes transthyretin amyloid deposits in plasma of the human subject by about 10% to about 99% (e.g., about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%) (e.g., when administered using any of the exemplary dosages and/or frequencies described herein).

Also provided herein are methods of reducing the level of transthyretin amyloid deposits in a human subject in need thereof including administering to a human subject in need thereof an amount of an antibody or an antigen-binding fragment thereof that reduces the level of transthyretin amyloid deposits, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof reduces the level of transthyretin amyloid deposits in plasma by greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99% (e.g., as compared to the level of transthyretin amyloid deposits in plasma of the human subject prior to the administering). In some embodiments, administering the antibody or the antigen-binding fragment thereof reduces the level of transthyretin amyloid deposits in plasma by about 10% to about 90%, about 20% to about 80%, about 30% to about 70%, about 40% to 60%, about 50% to about 99%, about 80% to about 99%, or about 90 to 99% (e.g., as compared to the level of transthyretin amyloid deposits in plasma of the human subject prior to the administering). In some embodiments, administering the antibody or the antigen-binding fragment thereof results in about a 10% to about a 99% decrease (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) in the level of transthyretin amyloid deposits in plasma in the human subject (e.g., as compared to the level of transthyretin amyloid deposits in the plasma in the human subject prior to administering) (e.g., when administered using any of the exemplary dosages and/or frequencies described herein).

Also provided herein are methods of inhibiting transthyretin amyloid fibril formation in a human subject in need thereof including administering to a human subject in need thereof an amount of an antibody or an antigen-binding fragment thereof that inhibits transthyretin amyloid fibril formation, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof inhibits transthyretin amyloid fibril formation in plasma by greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, administering the antibody or antigen-binding fragment thereof inhibits transthyretin amyloid fibril formation in plasma by about 10% to about 90%, about 20% to about 80%, about 30% to about 70%, about 40% to 60%, about 50% to about 99%, about 80% to about 99%, or about 90 to 99%. In some embodiments, administering the antibody or antigen-binding fragment thereof inhibits transthyretin amyloid fibril formation in plasma by about 10% to about 99% (e.g., about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%) (e.g., when administered using any of the exemplary dosages and/or frequencies described herein).

Also provided herein are methods of reducing the level of transthyretin amyloid fibrils in a human subject in need thereof including administering to a human subject in need thereof an amount of an antibody or an antigen-binding fragment thereof that reduces the level of transthyretin amyloid fibrils, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof reduces the level of transthyretin amyloid fibrils in plasma by greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, administering reduces the level of transthyretin amyloid fibrils in plasma by about 10% to about 90%, about 20% to about 80%, about 30% to about 70%, about 40% to 60%, about 50% to about 99%, about 80% to about 99%, or about 90 to 99% (e.g., as compared to the level of transthyretin amyloid fibrils in plasma of the human subject prior to the administering). In some embodiments, administering results in about a 10% to about a 99% decrease (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) in the level of transthyretin amyloid fibrils in plasma in the subject (e.g., as compared to the level of transthyretin amyloid fibrils in plasma of the human subject prior to the administering) (e.g., when administered using any of the exemplary dosages and/or frequencies described herein).

Also provided herein are methods of neutralizing soluble aggregate forms of misTTR in a human subject in need thereof including administering to a human subject in need thereof an amount of an antibody or an antigen-binding fragment thereof that neutralizes soluble aggregate forms of misTRR, wherein the antibody or the antigen-binding fragment thereof includes a heavy chain variable domain including complementarily determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment neutralizes soluble aggregate forms of misTTR in plasma by greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, administering the antibody or the antigen-binding fragment thereof neutralizes soluble aggregate forms of misTTR in plasma by about 10% to about 90%, about 20% to about 80%, about 30% to about 70%, about 40% to 60%, about 50% to about 99%, about 80% to about 99%, or about 90 to 99%. In some embodiments, administering the antibody or the antigen-binding fragment thereof results in about 10% to about 99% (e.g., about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%) neutralization of soluble aggregate forms of misTTR in plasma of the human subject (e.g., when administered using any of the exemplary dosages and/or frequencies described herein).

Also provided herein are methods increasing phagocytosis of insoluble transthyretin amyloid fibrils in a human subject in need thereof including administering to a human subject in need thereof an amount of an antibody or an antigen-binding fragment thereof that increases phagocytosis of insoluble transthyretin amyloid fibrils, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof increases phagocytosis of insoluble transthyretin amyloid fibrils in plasma by greater than about 20%, greater than about 25%, greater than about 30%, greater than about 35%, greater than about 40%, greater than about 45%, greater than about 50%, greater than about 55%, about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In some embodiments, administering the antibody or the antigen-binding fragment thereof increases phagocytosis of insoluble transthyretin amyloid fibrils in plasma by about 10% to about 90%, about 20% to about 80%, about 30% to about 70%, about 40% to 60%, about 50% to about 99%, about 80% to about 99%, or about 90 to 99%. In some embodiments, administering the antibody or the antigen-binding fragment thereof results in about a 10% to about a 99% increase (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% increase) in phagocytosis of insoluble transthyretin amyloid fibrils in plasma in the human subject (e.g., when administered using any of the exemplary dosages and/or frequencies described herein).

Also provided herein are methods of decreasing the level of insoluble transthyretin amyloid fibrils in a human subject in need thereof including administering to a human subject in need thereof an amount of an antibody or an antigen-binding fragment thereof that decreases the level of insoluble transthyretin amyloid fibrils, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or antigen-binding fragment thereof decreases the level of insoluble transthyretin amyloid fibrils in plasma by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% (e.g., as compared to the level of insoluble transthyretin amyloid fibrils in plasma of the human subject prior to administering). In some embodiments, administering the antibody or the antigen-binding fragment thereof decreases the level of insoluble transthyretin amyloid fibrils in plasma by about 10% to about 90%, about 20% to about 80%, about 30% to about 70%, about 40% to 60%, about 50% to about 99%, about 80% to about 99%, or about 90 to 99% (e.g., as compared to the level of insoluble transthyretin amyloid fibrils in plasma of the human subject prior to administering). In some embodiments, administering the antibody or the antigen-binding fragment thereof results in about a 10% to about a 99% decrease (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) in the level of insoluble transthyretin amyloid fibrils in plasma of the human subject (e.g., as compared to the level of insoluble amyloid transthyretin amyloid fibrils in plasma of the human subject prior to administering) (e.g., when administered using any of the exemplary dosages and/or frequencies described herein).

Also provided herein are methods of inhibiting formation of transthyretin amyloid deposits in a human subject in need thereof including administering to a human subject in need thereof an amount of an antibody or an antigen-binding fragment thereof that inhibits formation of transthyretin amyloid deposits, wherein the antibody or the antigen-binding fragment thereof includes a heavy chain variable domain including complementarily determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, administering the antibody or the antigen-binding fragment thereof inhibits the formation of transthyretin amyloid deposits in plasma by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%. In some embodiments, administering the antibody or the antigen-binding fragment thereof inhibits the formation of transthyretin amyloid deposits in plasma by about 10% to about 90%, about 20% to about 80%, about 30% to about 70%, about 40% to 60%, about 50% to about 99%, about 80% to about 99%, or about 90 to 99%. In some embodiments, administering the antibody or the antigen-binding fragment thereof inhibits the formation of transthyretin amyloid deposits in plasma in by about 10% to about 99% (e.g., about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 99%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 99%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 99%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 99%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 99%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 99%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 99%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 99%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 99%, about 55% to about 95%, about 55% to about 90%, about 55% to about 85%, about 55% to about 80%, about 55% to about 75%, about 55% to about 70%, about 55% to about 65%, about 55% to about 60%, about 60% to about 99%, about 60% to about 95%, about 60% to about 90%, about 60% to about 85%, about 60% to about 80%, about 60% to about 75%, about 60% to about 70%, about 60% to about 65%, about 65% to about 99%, about 65% to about 95%, about 65% to about 90%, about 65% to about 85%, about 65% to about 80%, about 65% to about 75%, about 65% to about 70%, about 70% to about 99%, about 70% to about 95%, about 70% to about 90%, about 70% to about 85%, about 70% to about 80%, about 70% to about 75%, about 75% to about 99%, about 75% to about 95%, about 75% to about 90%, about 75% to about 85%, about 75% to about 80%, about 80% to about 99%, about 80% to about 95%, about 80% to about 90%, about 80% to about 85%, about 85% to about 99%, about 85% to about 95%, about 85% to about 90%, about 90% to about 99%, about 90% to about 95%, or about 95% to about 99%) (e.g., when administered using any of the exemplary dosages and/or frequencies described herein).

Also provided herein are methods of reducing the level of transthyretin amyloid deposits in a human subject in need thereof including administering to a human subject in need thereof an amount of an antibody or an antigen-binding fragment thereof that reduces the level of transthyretin amyloid deposits, wherein the antibody or the antigen-binding fragment thereof includes a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, the administering of the antibody or the antigen-binding fragment thereof results in about a 10% to about a 99% decrease (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) in the level (e.g., plasma level) of transthyretin amyloid deposits in the human subject (e.g., as compared to the level (e.g., plasma level) of transthyretin amyloid deposits in the human subject prior to the administering) (e.g., when administered using any of the exemplary dosages and/or frequencies described herein).

Also provided herein are methods of treating amyloid transthyretin amyloidosis in a human subject in need thereof including administering to a human subject in need thereof a therapeutically effective amount of an antibody or an antigen-binding fragment thereof, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

PRX004 can also be used, for example, for diagnosing a TTR amyloidosis, for monitoring progression of a TTR amyloidosis, and/or for assessing efficacy of treatment. Such antibodies are particularly useful for performing such determinations in subjects having or being susceptible to a TTR amyloidosis, or in appropriate biological samples obtained from such subjects.

The terms “subject” or “patient” include human subjects that receive either prophylactic or therapeutic treatment, including treatment naïve individuals. Treatment naïve individuals may be naïve to PRX004 treatment or other treatments.

Provided herein are several methods of diagnosing, monitoring, treating or effecting prophylaxis of diseases or conditions mediated at least in part by transthyretin (TTR), and particularly by monomeric, misfolded, aggregated, or fibril forms of TTR (e.g., TTR amyloidosis). Examples of such diseases include familial TTR amyloidoses, such as familial amyloid cardiomyopathy (FAC) (e.g., ATTR-CM) or cardiomyopathy or hypertrophy in athletes or others undergoing extreme aerobic exercise, familial amyloid polyneuropathy (FAP), or central nervous system selective amyloidosis (CNSA), and sporadic TTR amyloidoses, such as senile systemic amyloidosis (SSA) or senile cardiac amyloidosis (SCA). TTR amyloidosis can also be associated as a cause or result of various diseases and conditions characterized by tissue or organ degeneration or trauma. Accumulation of TTR deposits contributes to organ or tissue dysfunction associated with the disease or condition. An example of such a condition amenable to treatment or prophylaxis with the present agents and methods is spinal stenosis (Westermark et al., Upsala J. Medical Sciences 119, 223-238 (2014) and Yanagisawa et al., Modern Pathology 28, 201-207 (2015). Another disease likewise amenable to treatment or prophylaxis is osteoarthritis (Takanashi et al., Amyloid 20, 151-155 (2013), Gu et al., Biomed & Biotechnol. 15, 92-99; Takinami et al., Biomarker Insights 8, 85-95 (2014); Akasaki et al., Arthritis Rheumatol. 67, 2097-2107 (2015). Another disease likewise amenable to treatment or prophylaxis is rheumatoid arthritis (Clement et al., JCI Insight 1 epublish (2016). Another disease amenable to treatment or prophylaxis is juvenile idiopathic arthritis (Sharma et al., PLOSone 9, 1-12 (2014). Another disease amenable to treatment or prophylaxis is age related macular degeneration (wet or dry). Another class of conditions likewise amenable to treatment or prophylaxis are ligament and tendon disorders, such as disorders of the rotator cuff (Sueyoshi et al,. Human Pathol. 42, 1259-64 (2011).

In one embodiment, antibodies described above (e.g., PRX004) can be incorporated into a composition for suitable for administration to a subject in need thereof. In another embodiment, antibodies described above (e.g., PRX004) can be incorporated into a pharmaceutical composition for use of treatment or prophylaxis of any of the above diseases and conditions. In general, an antibody or pharmaceutical composition containing an antibody is administered to a subject in need thereof. Patients amenable to treatment include individuals at risk of TTR amyloidosis but not showing symptoms, as well as patients presently showing symptoms. Some patients can be treated during the prodromal stage of TTR amyloidosis.

Individuals suffering from TTR amyloidosis can sometimes be recognized from the clinical manifestations of TTR amyloidosis, including one or more of the following: (1) family history of neuropathic disease, especially associated with heart failure; (2) neuropathic pain or progressive sensory disturbances of unknown etiology; (3) carpal tunnel syndrome without obvious cause, particularly if it is bilateral and requires surgical release; (4) gastrointestinal motility disturbances or autonomic nerve dysfunction of unknown etiology (e.g., erectile dysfunction, orthostatic hypotension, neurogenic gladder); (5) cardiac disease characterized by thickened ventricular walls in the absence of hypertension; (6) advanced atrio-ventricular block of unknown origin, particularly when accompanied by a thickened heart; and (6) vitreous body inclusions of the cotton-wool type. See Ando et al., Orphanet Journal of Rare Diseases 8:31 (2013). Definitive diagnosis of TTR amyloidosis, however, typically relies on target organ biopsies, followed by histological staining of the excised tissue with the amyloid-specific dye, Congo red. If a positive test for amyloid is observed, immunohistochemical staining for TTR is subsequently performed to ensure that the precursor protein responsible for amyloid formation is indeed TTR. For familial forms of the diseases, demonstration of a mutation in the gene encoding TTR is then needed before a definitive diagnosis can be made.

The identification of the subject can occur in a clinical setting, or elsewhere, such as in the subject's home, for example, through the subject's own use of a self-testing kit. For example, the subject can be identified based on various symptoms such as peripheral neuropathy (sensory and motor), autonomic neuropathy, gastrointestinal impairment, cardiomyopathy, nephropathy, or ocular deposition. See Ando et al., Orphanet Journal of Rare Diseases 8:31 (2013). The subject can also be identified by increased levels of non-native forms of TTR in plasma samples from the subject compared to control samples, as disclosed in the examples.

As warranted by family history, genetic testing, or medical screening for TTR amyloidosis, treatment can begin at any age (e.g., 20, 30, 40, 50, 60, or 70 years of age). Treatment typically entails multiple dosages over a period of time and can be monitored by assaying antibody or activated T-cell or B-cell responses to a therapeutic agent (e. g., a truncated form of TTR comprising amino acid residues 89-97) over time. If the response falls, a booster dosage is indicated.

In prophylactic applications, an antibody or a pharmaceutical composition of the same is administered to a subject susceptible to, or otherwise at risk of a disease (e.g., TTR amyloidosis) in a regime (dose, frequency and route of administration) effective to reduce the risk, lessen the severity, or delay the onset of at least one sign or symptom of the disease. In therapeutic applications, an antibody or immunogen to induce an antibody is administered to a subject suspected of, or already suffering from a disease (e.g., TTR amyloidosis) in a regime (dose, frequency and route of administration) effective to ameliorate or at least inhibit further deterioration of at least one sign or symptom of the disease.

Also provided herein are methods of improving the neuropathy in a human subject with ATTR amyloidosis in need thereof including administering to a human subject in need thereof a therapeutically effective amount of an antibody or an antigen-binding fragment thereof, where the antibody or the antigen-binding fragment thereof comprises a heavy chain variable domain including complementarily determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, the administering results in a reduction of the neuropathy impairment score (NIS).

Also provided herein are methods of treating ATTR amyloidosis in a human subject in need thereof including administering to a human subject in need thereof a therapeutically effective amount of an antibody or an antigen-binding fragment thereof where the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6, wherein the administering reduces the NIS in the human subject in need thereof.

In some embodiments, the administering of the antibody or the antigen-binding fragment thereof results in a reduced NIS of about 1 to about 50 (e.g., about 1 to about 45, about 1 to about 40, about 1 to about 35, about 1 to about 30, about 1 to about about 1 to about 20, about 1 to about 15, about 1 to about 10, about 1 to about 5, about 5 to about 50, about 5 about 45, about 5 to about 40, about 5 to about 35, about 5 to about 30, about 5 to about 25, about 5 to about 20, about 5 to about 15, about 5 to about 10, about 15 to about 50, about 15 to about 45, about 15 to about 40, about 15 to about 35, about 15 to about 30, about 15 to about 25, about 15 to about 20, about 20 to about 50, about 25 to about 45, about 20 to about 40, about 20 to about 35, about 20 to about 30, about 20 to about 25, about 25 to about 50, about 25 to about 45, about 25 to about 40, about 25 to about 35, about 25 to about 30, about 30 to about 50, about 30 to about 45, about 30 to about 40, about 30 to about 35, about 35 to about 50, about 35 to about 45, about 35 to about 40, about 40 to about 50, or about 45 to about 50) (e.g., as compared to the NIS in the human subject prior to the administering (e.g., when administered using any of the exemplary dosages and/or frequencies described herein).

In some embodiments, the administering of the antibody or the antigen-binding fragment thereof results in a reduced NIS of about OA to about 5.0 (e.g., about to about 5, about 0.1 to about 4.9, about 0.1 to about 4.8, about 0.1 to about 4.7, about 0.1 to about 4.6, about 0.1 to about 4.5, about 0.1 to about 4.4, about 0.1 to about 4.3, about 0.1 to about 4.2, about 0.1 to about 4.1, about 0.1 to about 4, about 0.1 to about 3.9, about 0.1 to about 3.8, about 0.1 to about 3.7, about 0.1 to about 3.6, about 0.1 to about 3.5, about 0.1 to about 3.4, about 0.1 to about 3.3, about 0.1 to about 3.2, about to about 3.1, about 0.1 to about 3, about 0.1 to about 2.9, about 0.1 to about 2.8, about 0.1 to about 2.7, about 0.1 to about 2.6, about 0.1 to about 2.5, about 0.1 to about 2.4, about 0.1 to about 2.3, about 0.1 to about 2.2, about 0.1 to about 2.1, about 0.1 to about 2, about 0.1 to about 1.9, about 0.1 to about 1.8, about 0.1 to about 1.7, about 0.1 to about 1.6, about 0.1 to about 1.5, about 0.1 to about 1.4, about 0.1 to about 1.3, about to about 1.2, about 0.1 to about 1.1, about 0.1 to about 1, about 0.1 to about 0.9, about 0.1 to about 0.8, about 0.1 to about 0.7, about 0.1 to about 0.6, about 0.1 to about about 0.1 to about 0.4, about 0.1 to about 0.3, about 0.1 to about 0.2, about 0.5 to about 5, about 0.5 to about 4.9, about 0.5 to about 4.8, about 0.5 to about 4.7, about 0.5 to about 4.6, about 0.5 to about 4.5, about 0.5 to about 4.4, about 0.5 to about 4.3, about to about 4.2, about 0.5 to about 4.1, about 0.5 to about 4, about 0.5 to about 3.9, about 0.5 to about 3.8, about 0.5 to about 3.7, about 0.5 to about 3.6, about 0.5 to about 3.5, about 0.5 to about 3.4, about 0.5 to about 3.3, about 0.5 to about 3.2, about 0.5 to about 3.1, about 0.5 to about 3, about 0.5 to about 2.9, about 0.5 to about 2.8, about 0.5 to about 2.7, about 0.5 to about 2.6, about 0.5 to about 2.5, about 0.5 to about 2.4, about to about 2.3, about 0.5 to about 2.2, about 0.5 to about 2.1, about 0.5 to about 2, about 0.5 to about 1.9, about 0.5 to about 1.8, about 0.5 to about 1.7, about 0.5 to about 1.6, about 0.5 to about 1.5, about 0.5 to about 1.4, about 0.5 to about 1.3, about 0.5 to about 1.2, about 0.5 to about 1.1, about 0.5 to about 1, about 0.5 to about 0.9, about 0.5 to about 0.8, about 0.5 to about 0.7, about 0.5 to about 0.6, about 1 to about 5, about 1 to about 4.9, about 1 to about 4.8, about 1 to about 4.7, about 1 to about 4.6, about 1 to about 4.5, about 1 to about 4.4, about 1 to about 4.3, about 1 to about 4.2, about 1 to about 4.1, about 1 to about 4, about 1 to about 3.9, about 1 to about 3.8, about 1 to about 3.7, about 1 to about 3.6, about 1 to about 3.5, about 1 to about 3.4, about 1 to about 3.3, about 1 to about 3.2, about 1 to about 3.1, about 1 to about 3, about 1 to about 2.9, about 1 to about 2.8, about 1 to about 2.7, about 1 to about 2.6, about 1 to about 2.5, about 1 to about 2.4, about 1 to about 2.3, about 1 to about 2.2, about 1 to about 2.1, about 1 to about 2, about 1 to about 1.9, about 1 to about 1.8, about 1 to about 1.7, about 1 to about 1.6, about 1 to about 1.5, about 1 to about 1.4, about 1 to about 1.3, about 1 to about 1.2, about 1 to about 1.1, about 1.5 to about 5, about 1.5 to about 4.9, about 1.5 to about 4.8, about 1.5 to about 4.7, about 1.5 to about 4.6, about 1.5 to about 4.5, about 1.5 to about 4.4, about 1.5 to about 4.3, about 1.5 to about 4.2, about 1.5 to about 4.1, about 1.5 to about 4, about 1.5 to about 3.9, about 1.5 to about 3.8, about 1.5 to about 3.7, about 1.5 to about 3.6, about 1.5 to about 3.5, about 1.5 to about 3.4, about 1.5 to about 3.3, about 1.5 to about 3.2, about 1.5 to about 3.1, about 1.5 to about 3, about 1.5 to about 2.9, about 1.5 to about 2.8, about 1.5 to about 2.7, about 1.5 to about 2.6, about 1.5 to about 2.5, about 1.5 to about 2.4, about 1.5 to about 2.3, about 1.5 to about 2.2, about 1.5 to about 2.1, about 1.5 to about 2, about 1.5 to about 1.9, about 1.5 to about 1.8, about 1.5 to about 1.7, about 1.5 to about 1.6, about 2 to about 5, about 2 to about 4.9, about 2 to about 4.8, about 2 to about 4.7, about 2 to about 4.6, about 2 to about 4.5, about 2 to about 4.4, about 2 to about 4.3, about 2 to about 4.2, about 2 to about 4.1, about 2 to about 4, about 2 to about 3.9, about 2 to about 3.8, about 2 to about 3.7, about 2 to about 3.6, about 2 to about 3.5, about 2 to about 3.4, about 2 to about 3.3, about 2 to about 3.2, about 2 to about 3.1, about 2 to about 3, about 2 to about 2.9, about 2 to about 2.8, about 2 to about 2.7, about 2 to about 2.6, about 2 to about 2.5, about 2 to about 2.4, about 2 to about 2.3, about 2 to about 2.2, about 2 to about 2.1, about 2.5 to about 5, about 2.5 to about 4.9, about 2.5 to about 4.8, about 2.5 to about 4.7, about 2.5 to about 4.6, about 2.5 to about 4.5, about 2.5 to about 4.4, about 2.5 to about 4.3, about 2.5 to about 4.2, about 2.5 to about 4.1, about 2.5 to about 4, about 2.5 to about 3.9, about 2.5 to about 3.8, about 2.5 to about 3.7, about 2.5 to about 3.6, about 2.5 to about 3.5, about 2.5 to about 3.4, about 2.5 to about 3.3, about 2.5 to about 3.2, about 2.5 to about 3.1, about 2.5 to about 3, about 2.5 to about 2.9, about 2.5 to about 2.8, about 2.5 to about 2.7, about 2.5 to about 2.6, about 3 to about 5, about 3 to about 4.9, about 3 to about 4.8, about 3 to about 4.7, about 3 to about 4.6, about 3 to about 4.5, about 3 to about 4.4, about 3 to about 4.3, about 3 to about 4.2, about 3 to about 4.1, about 3 to about 4, about 3 to about 3.9, about 3 to about 3.8, about 3 to about 3.7, about 3 to about 3.6, about 3 to about 3.5, about 3 to about 3.4, about 3 to about 3.3, about 3 to about 3.2, about 3 to about 3.1, about 3.5 to about 5, about 3.5 to about 4.9, about 3.5 to about 4.8, about 3.5 to about 4.7, about 3.5 to about 4.6, about 3.5 to about 4.5, about 3.5 to about 4.4, about 3.5 to about 4.3, about 3.5 to about 4.2, about 3.5 to about 4.1, about 3.5 to about 4, about 3.5 to about 3.9, about 3.5 to about 3.8, about 3.5 to about 3.7, about 3.5 to about 3.6, about 4 to about 5, about 4 to about 4.9, about 4 to about 4.8, about 4 to about 4.7, about 4 to about 4.6, about 4 to about 4.5, about 4 to about 4.4, about 4 to about 4.3, about 4 to about 4.2, about 4 to about 4.1, about 4.5 to about 5, about 4.5 to about 4.9, about 4.5 to about 4.8, about 4.5 to about 4.7, or about 4.5 to about 4.6 (e.g., as compared to the NIS in the human subject prior to the administering (e.g., when administered using any of the exemplary dosages and/or frequencies described herein).

In some embodiments, the administering of the antibody or the antigen-binding fragment thereof results in a reduction in NIS of at least about 0.1, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 (e.g., as compared to the NIS in the human subject prior to the administering (e.g., when administered using any of the exemplary dosages and/or frequencies described herein).

In some embodiments, the administering of the antibody or the antigen-binding fragment thereof results in a reduction in NIS of at least about 0.1, at least about at least about 0.3, at least about 0.4, at least about 0.5, at least about 0.6, at least about 0.7, at least about 0.8, at least about 0.9, at least about 1, at least about 1.1, at least about 1.2, at least about 1.3, at least about 1.4, at least about 1.5, at least about 1.6, at least about 1.7, at least about 1.8, at least about 1.9, at least about 2, at least about 2.1, at least about 2.2, at least about 2.3, at least about 2.4, at least about 2.5, at least about 2.6, at least about 2.7, at least about 2.8, at least about 2.9, at least about 3, at least about 3.1, at least about 3.2, at least about 3.3, at least about 3.4, at least about 3.5, at least about 3.6, at least about 3.7, at least about 3.8, at least about 3.9, at least about 4, at least about 4.1, at least about 4.2, at least about 4.3, at least about 4.4, at least about 4.5, at least about 4.6, at least about 4.7, at least about 4.8, at least about 4.9, or at least about 5 (e.g., as compared to the NIS in the human subject prior to the administering (e.g., when administered using any of the exemplary dosages and/or frequencies described herein).

Also provided herein are methods of improving cardiac systolic function in a human subject with ATTR amyloidosis in need thereof including administering to a human subject in need thereof a therapeutically effective amount of an antibody or an antigen-binding fragment thereof, where the antibody or the antigen-binding fragment thereof comprises a heavy chain variable domain including complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

In some embodiments, the administering results in reducing the global longitudinal strain (GLS) in a human subject.

Also provided herein are methods of treating ATTR amyloidosis in a human subject in need thereof including administering to a human subject in need thereof a therapeutically effective amount of an antibody or an antigen-binding fragment thereof where the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6, wherein the administering reduces the global longitudinal strain (GLS) in the human subject in need thereof.

In some embodiments, the administering of the antibody or the antigen-binding fragment thereof results in an improved cardiac systolic function as measured by global longitudinal strain (GLS) of about 0.1% to about 99% (e.g., about 0.1% to about 99%, about 0.1% to about 95%, about 0.1% to about 90%, about 0.1% to about 85%, about 0.1% to about 80%, about 0.1% to about 75%, about 0.1% to about 70%, about to about 65%, about 0.1% to about 60%, about 0.1% to about 55%, about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, about 0.1% to about 10%, about 0.1% to about 5%, about to about 1%, about 1% to about 99%, about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 99%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to the GLS in the human subject prior to the administering (e.g., when administered using any of the exemplary dosages and/or frequencies described herein).

In some embodiments, the administering of the antibody or the antigen-binding fragment thereof results in an improved cardiac systolic function as measured by global longitudinal strain (GLS) of at least about at least about 0.5%, at least about 1%, at least about 1.5%, at least about 2%, at least about 2.5%, at least about 3%, at least about 3.5%, at least about 4%, at least about 4.5%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%) (e.g., when administered using any of the exemplary dosages and/or frequencies described herein).

In some embodiments, the administering of the antibody or the antigen-binding fragment thereof results in an improved cardiac systolic function as measured by GLS of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.1%, at least about 3.2%, at least about 3.3%, at least about 3.4%, at least about 3.5%, at least about 3.6%, at least about 3.7%, at least about 3.8%, at least about 3.9%, at least about 4%, at least about 4.1%, at least about 4.2%, at least about 4.3%, at least about 4.4%, at least about 4.5%, at least about 4.6%, at least about 4.7%, at least about 4.8%, at least about 4.9%, or at least about 5%) (e.g., when administered using any of the exemplary dosages and/or frequencies described herein).

Also provided herein are method of treating heart failure in a human subject with ATTR amyloidosis in need thereof including administering to a human subject in need thereof a therapeutically effective amount of an antibody or an antigen-binding fragment thereof, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6, and the New York Heart Association (NYHA) class of the human subject in need thereof does not increase.

Generally, subjects' heart failure is classified according to the severity of their symptoms. The New York Heart Association (NYHA) classification system includes four classes (I, II, III, and IV). Subject symptoms associated with each class are listed below.
Class I: No limitation of physical activity. Ordinary physical activity does not cause undue fatigue, palpitation, dyspnea (shortness of breath).
Class II: Slight limitation of physical activity. Comfortable at rest. Ordinary physical activity results in fatigue, palpitation, dyspnea (shortness of breath).
Class III: Marked limitation of physical activity. Comfortable at rest. Less than ordinary activity causes fatigue, palpitation, or dyspnea.
Class IV: Unable to carry on any physical activity without discomfort. Symptoms of heart failure at rest. If any physical activity is undertaken, discomfort increases.

Thus, in some embodiments, of treating amyloid transthyretin amyloidosis, administering a therapeutically effective amount of antibody or an antigen-binding fragment described herein does not result in an increase in the NYHA class after a period of time sufficient to treat NYHA class I, II, or III in the human subject.

For example, a subject with a baseline NYHA class determination of class I, remains at NYHA class I after a period of time sufficient to treat the subject, rather than progressing to the next NYHA class or beyond.

Also provided herein are methods of treating heart failure in a human subject with ATTR amyloidosis in need thereof including administering to a human subject in need thereof a therapeutically effective amount of an antibody or an antigen-binding fragment thereof, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6, and the administering results in an improved NYHA class after a period of time sufficient to treat NYHA class I, II, III, or IV in the human subject.

For example, a subject with a baseline NYHA class determination of class II, improves to NYHA class I after a period of time sufficient to treat the subject (e.g., a period of time as described herein), rather than remaining at class II or progressing to the next NYHA class or beyond when assessed at a later time point. Other possible examples (e.g., a subject moving from class III to class II) are understood by way of the previous example.

Also provided herein are nucleic acids encoding any of the heavy and light chains described above (e.g., SEQ ID NOS: 7-10). Coding sequences of nucleic acids can be operably linked with regulatory sequences to ensure expression of the coding sequences, such as a promoter, enhancer, ribosome binding site, transcription termination signal, and the like. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

In some embodiments of the methods described herein, PRX004 is administered to a human subject between about 0.1 mg/kg to about 50 mg/kg of PRX004. In some embodiments of the methods described herein, PRX004 is administered to the human subject between about 1 mg/kg to about 30 mg/kg of PRX004. In some embodiments of the methods described herein, PRX004 is administered to the human subject includes between about 3 mg/kg to about 10 mg/kg or about 10 mg/kg to about 30 mg/kg. In some embodiments of the methods described herein, PRX004 is administered to a human subject at about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, or about 50 mg/kg. In some embodiments, PRX004 is administered to a human subject between about 0.1 mg/kg, about 0.3 mg/kg, about 1.0 mg/kg, about 3 mg/kg, about 10 mg/kg, or about 30 mg/kg of PRX004.

In some embodiments of the methods described herein, PRX004 is administered to the subject intravenously.

In some embodiments of the methods described herein, PRX004 is administered to the subject in need thereof indefinitely. In some embodiments of the methods described herein, PRX004 is administered between about 1 month to about 1 year, about 2 months to about 11 months, about 3 months to about 10 months, about 4 months to about 9 months, or about 5 months to about 8 months. In some embodiments, PRX004 is administered for about 12 months, 18 months, about 24 months, about 30 months, about 36 months, about 42 months, about 48 months, about 54 months, about 60 months, about 66 months, about 72 months, about 78 months, about 84 months, about 90 months, about 96 months, about 102 months, about 108 months, about 114 months, or about 120 months.

In some embodiments of the methods described herein, PRX004 is administered to the subject in need thereof at an interval (or dosing frequency) of about every 14 days, about every 2 weeks, about every 3 weeks, about every 28 days, about every 4 weeks, about monthly, about every 5 weeks, about every 6 weeks, about every 7 weeks, about every 8 weeks, or about every 2 months.

Also provided herein are kits including an antibody or an antigen-binding fragment thereof, where the antibody or the antigen-binding fragment thereof includes a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6; and instructions for performing any of the methods described herein.

In some kits, the antibody or the antigen-binding fragment thereof comprises a heavy chain variable domain comprising SEQ ID NO: 7 and a light chain variable domain comprising SEQ ID NO: 8.

In some kits, the antibody or the antigen-binding fragment thereof comprises a heavy chain comprising SEQ ID NO: 9 and a light chain comprising SEQ ID NO: 10. In some embodiments, the antibody or the antigen-binding fragment thereof does not comprise a C-terminal lysine.

### EXAMPLES

### Example 1. Evaluation of PRX004 Antibody

In this Phase 1, open-label, multicenter, 3+3 dose-escalation study (NCT03336580), 21 patients with hATTR were enrolled in 4 countries to receive PRX004 intravenously once every 28 days for up to 3 infusions in the dose-escalation (DE) phase. Patients were enrolled into one of the following 6 PRX004 dose cohorts: 0.1, 0.3, 1, 3, 10, and 30 mg/kg, starting with the lowest dose. Enrollment in the next higher dose levels occurred when no dose limiting toxicities (DLTs) were observed. Eligible patients received up to 15 additional infusions in the long-term extension (LTE) phase. Stable doses of tafamidis or diflusinal were allowed.

For this phase 1 study the following objectives were evaluated. Primary objectives: evaluate safety, tolerability, PK and target engagement (misTTR assay), Determine MTD or RP2D(s). Secondary objective: evaluate immunogenicity. Exploratory objectives: characterize efficacy (NIS) in patients with hATTR-PN with or without hATTR-CM (Cohorts 4-6).

Of the 21 study patients, the majority were male and Caucasian, ranging from 42 to 78 years old. Three patients had cardiomyopathy (CM) only, 7 had peripheral neuropathy (PN) only, and 11 had both CM and PN (Table 1).

TABLE 1
Baseline characteristics of study patients

| | PRX004 | | PRX004 | | PRX004 | | PRX004 | | PRX004 | | PRX004 | | | |
|---|---|---|---|---|---|---|---|---|---|---|---|---|---|---|
| | 0.1 mg/kg | | 0.3 mg/kg | | 1 mg/kg | | 3 mg/kg | | 10 mg/kg | | 30 mg/kg | | Overall | |
| | (N = 3) | | (N = 3) | | (N = 3) | | (N = 3) | | (N = 3) | | (N = 6) | | (N = 21) | |
| | n (%) | | n (%) | | n (%) | | n (%) | | n (%) | | n (%) | | n (%) | |
| Age Range | 68-72 | | 65-68 | | 66-75 | | 58-78 | | 42-75 | | 44-70 | | 42-78 | |
| Sex: | | | | | | | | | | | | | | |
| Male | 2 | (66.7%) | 1 | (33.3%) | 2 | (66.7%) | 2 | (66.7%) | 1 | (33.3%) | 5 | (83.3%) | 13 | (61.9%) |
| Female | 1 | (33.3%) | 2 | (66.7%) | 1 | (33.3%) | 1 | (33.3%) | 2 | (66.7%) | 1 | (16.7) | 8 | (38.1%) |
| Race: | | | | | | | | | | | | | | |
| White | 3 | (100%) | 3 | (100%) | 3 | (100%) | 3 | (100%) | 3 | (100%) | 5 | (83.3%) | 20 | (95.2%) |
| Black or African | 0 | (0) | 0 | (0) | 0 | (0) | 0 | (0) | 0 | (0) | 1 | (16.7%) | 1 | (4.8%) |
| American | | | | | | | | | | | | | | |
| Ethnicity: | | | | | | | | | | | | | | |
| Not Hispanic or | 3 | (100%) | 3 | (100%) | 3 | (100%) | 3 | (100%) | 3 | (100%) | 6 | (100%) | 21 | (100%) |
| Latino | | | | | | | | | | | | | | |
| Primary Disease*: | | | | | | | | | | | | | | |
| Cardiomyopathy | N/A | | 1 | (33.3%) | 2 | (66.7%) | N/A | | N/A | | N/A | | 3 | (14.3%) |
| only | | | | | | | | | | | | | | |
| Peripheral | 1 | (33.3%) | 1 | (33.3%) | N/A | | 2 | (66.7%) | 2 | (66.7%) | 1 | (16.7%) | 7 | (33.3%) |
| Neuropathy only | | | | | | | | | | | | | | |
| Both CM and PN | 2 | (66.7%) | 1 | (33.3%) | 1 | (33.3%) | 1 | (33.3%) | 1 | (33.3%) | 5 | (83.3%) | 11 | (52.4%) |

*Defined per the Principal Investigator's clinical impression

All 21 patients completed the DE phase and 17 subsequently enrolled in the LTE, receiving between 1 to 14 additional doses of PRX004. No drug-related serious adverse events (AEs), drug-related≥Grade 3 events, deaths, or DLTs were reported in either the DE or LTE phase of the study. The most frequent TEAEs≥10% were fall, anemia, constipation, upper respiratory tract infection; back pain, diarrhea, and insomnia. (Table 2).

TABLE 2
Overall summary of treatment emergent adverse events

| | PRX004 | PRX004 | PRX004 | PRX004 | PRX004 | PRX004 | | |
|---|---|---|---|---|---|---|---|---|
| | 0.1 mg/kg | 0.3 mg/kg | 1 mg/kg | 3 mg/kg | 10 mg/kg | 30 mg/kg | Overall | |
| | (N = 3) | (N = 3) | (N = 3) | (N = 3) | (N = 3) | (N = 6) | (N = 21) | |
| | n (%) | n (%) | n (%) | n (%) | n (%) | n (%) | n (%) | |
| Total Number of TEAEs | 2 | 23 | 53 | 31 | 19 | 39 | 167 | |
| Number (%) of Subjects | | | | | | | | |
| Reporting at Least One: | | | | | | | | |
| TEAE | 1 (33.3%) | 3 (100%) | 3 (100%) | 3 (100%) | 3 (100%) | 5 (83.3%) | 18 | (85.7%) |
| TEAE by Maximum | | | | | | | | |
| CTCAE Grade: | | | | | | | | |
| Grade 1 - Mild | 0 | 2 (66.7%) | 0 | 0 | 0 | 1 (16.7%) | 3 | (14.3%) |
| Grade 2 - Moderate | 1 (33.3%) | 1 (33.3%) | 2 (66.7%) | 1 (33.3%) | 1 (33.3%) | 3 (50.0%) | 9 | (42.9%) |
| Grade 3 - Severe | 0 | 0 | 1 (33.3%) | 2 (66.7%) | 2 (66.7%) | 1 (16.7%) | 6 | (28.6%) |
| Grade 4 - Life- | 0 | 0 | 0 | 0 | 0 | 0 | 0 | |
| Threatening | | | | | | | | |
| Grade 5 - Fatal | 0 | 0 | 0 | 0 | 0 | 0 | 0 | |
| TEAE by Relationship: | | | | | | | | |
| Not Related | 1 (33.3%) | 3 (100%) | 2 (66.7%) | 3 (100%) | 2 (66.7%) | 1 (16.7%) | 12 | (57.1%) |
| Related | 0 | 0 | 1 (33.3%) | 0 | 1 (33.3%) | 4 (66.7%) | 6 | (28.6%) |
| TEAE Leading to Study | 0 | 0 | 0 | 0 | 1 (33.3%) | 0 | 1 | (4.8%) |
| Drug Withdrawal | | | | | (Pregnancy) | | | |
| Total Number of Serious | 0 | 0 | 1 (33.3%) | 0 | 2 (66.7%) | 2 (33.3%) | 5 | (23.8%) |
| TEAEs | | | | | | | | |
| Serious TEAE by | | | | | | | | |
| Relationship: | | | | | | | | |
| Not Related | 0 | 0 | 1 (33.3%) | 0 | 2 (66.7%) | 2 (33.3%) | 5 | (23.8%) |

| | | | | | | | |
|---|---|---|---|---|---|---|---|
| Related | 0 | 0 | 0 | 0 | 0 | 0 | 0 |

Two subjects had transient positive anti-drug antibodies, however, there were no clinical sequalae in these subjects and no change was noted on PK.

The LTE portion of the study was ongoing when the COVID-19 pandemic began. As a result of the pandemic, patients missed study visits in the long-term extension and this portion of the study was ultimately discontinued. As a result, the 7 patients in cohorts 4, 5 and 6 of the Phase 1 study that received all infusions through 9 months were considered evaluable for efficacy.

Pharmacokinetics Analysis of PRX004

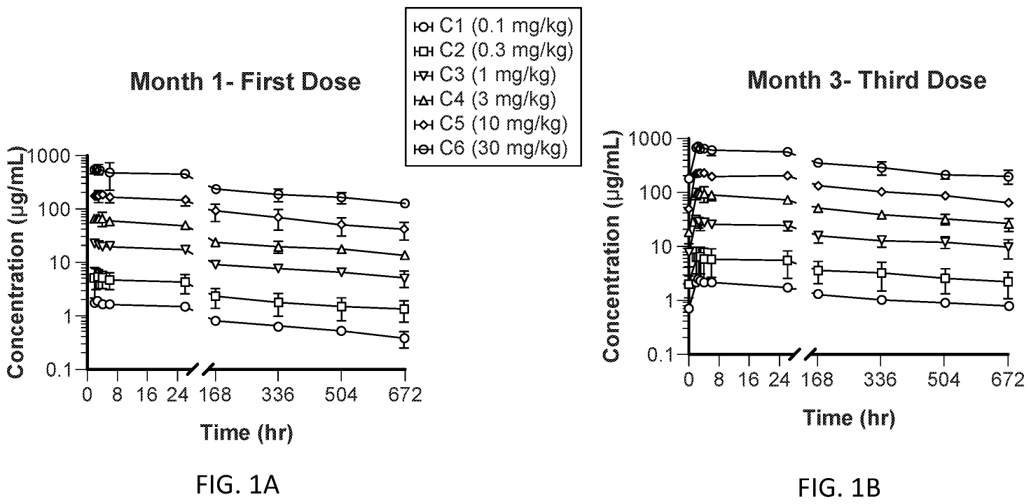

PRX004 demonstrated a PK profile consistent with IgG1 monoclonal antibodies, and exposures increased proportionally with dose. FIGS. 1A and 1B show the concentration (Kg/ml) of PRX004 over time at the first dose in month 1 (FIG. 1A) and the dose at month 3 (FIG. 1B) for cohorts 1-6 (0.1 mg/kg, 0.3 mg/kg, 1 mg/kg, 3 mg/kg, 10 mg/kg, and 30 mg/kg, respectively). Table 3 shows the pharmacokinetic data at Month 3. The data demonstrate that exposure to the antibody increase in a dose proportional manner. The mean observed T112 of approximately 31 days was similar across all six dose level cohorts.

Validation of PRX004 Dose Selection

Since no quantitative non-invasive measure of amyloid levels in patients currently exists, we utilized a measure of the binding of PRX004 to misfolded TTR species in the plasma of patients using our proprietary assay for misfolded TTR.

To evaluate expected amyloid binding several variables were factored into the analysis, including reduction in free non-native TTR by PRX004, accounting for plasma dilution and binding kinetics to amyloid vs. plasma monomers, and antibody exposure in heart. For instance, these non-native plasma species were composed mainly of monomers and are likely the direct product of TTR dissociation in blood. PRX004 binds to monomers with lower affinity than to amyloid. In addition, preanalytical conditions specific to the assay result in dissociation of PRX004 after the plasma is collected. When these factors are accounted for we can accurately estimate amyloid binding.

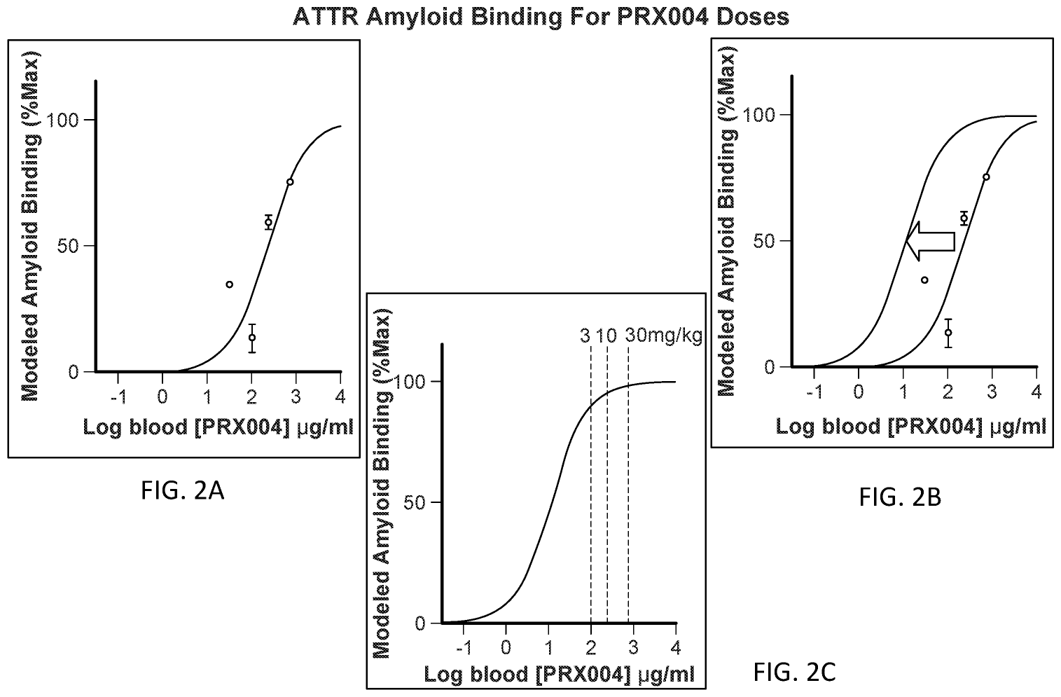

The resulting model is illustrated in FIGS. 2A-C which demonstrate that dose levels of PRX004 equal to or greater than 3 mg/kg are equivalent and sufficient to achieve saturation in tissue. FIG. 2A shows the results of the modeled amyloid binding based on the binding of PRX004 to misfolded TTR from in different dose cohorts. FIG. 2B shows the adjustment to the curve (red line shifted left) based on the factors described above. FIG. 2C overlays the concertation of PRX004 in blood (μg/mL) for different doses to show that PRX004 doses≥3 mg/kg are expected to reach exposures to occupy >90% of amyloid. Based on these findings, we pooled cohorts 4, 5, and 6 (doses of 3 mg/kg, 10 mg/kg, and 30 mg/kg, respectively) for efficacy assessments.

Target Engagement of PRX004

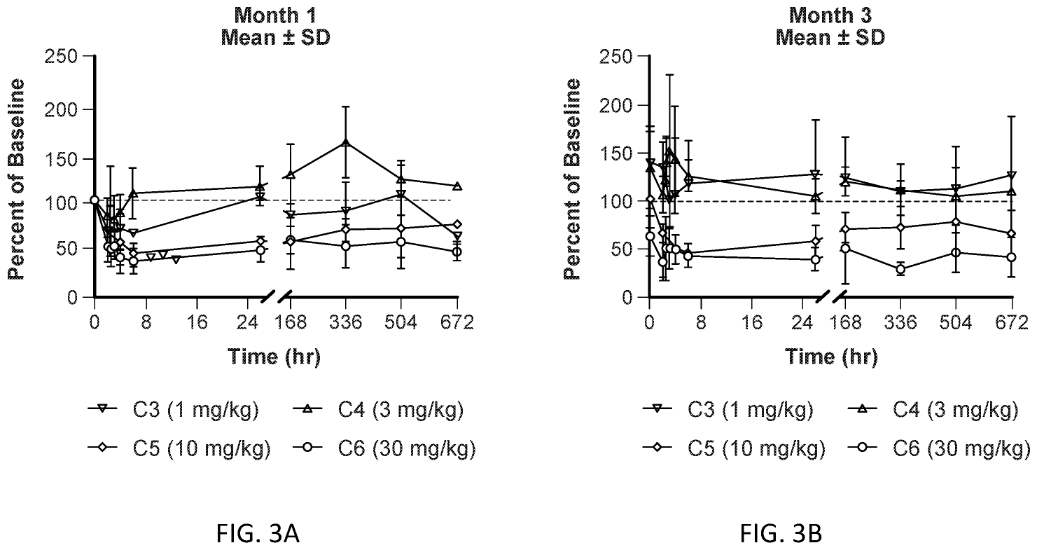

Target engagement was demonstrated by a time and dose-dependent decrease in plasma levels of unbound misTTR. FIGS. 3A-B are graphs showing the amount of misTTR as the percent of baseline over time after administration of PRX004 in month 1 (FIG. 3A) and after the dose administered in month 3 (FIG. 3B) for cohorts 3, 4, 5, and 6.

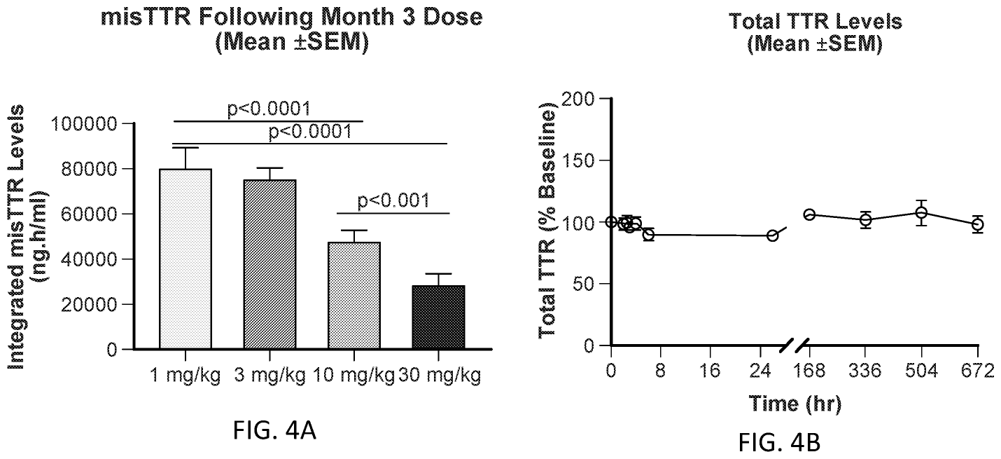

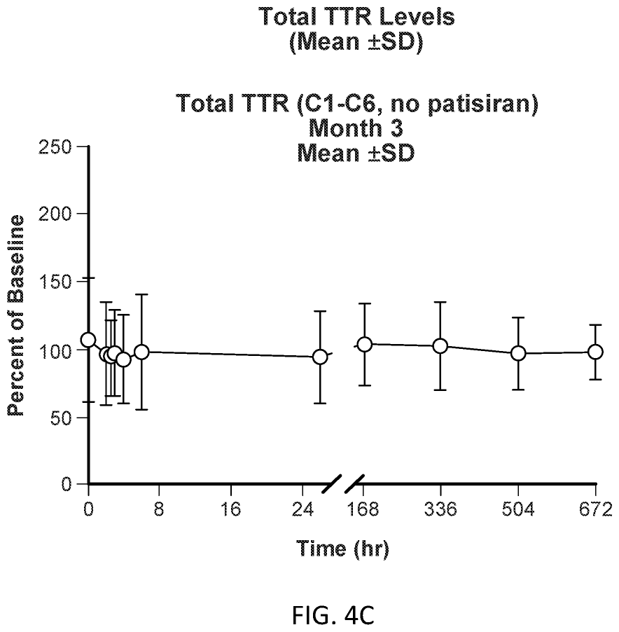

FIG. 4A is graph showing the dose-dependent decrease in misTTR for cohorts 3, 4, 5, and 6. Reduction of free misTTR provides evidence of target engagement. Cohorts 3 and 4 (1 mg/kg and 3 mg/kg) baseline normalization may be impacted by residual effect of patisiran; Cohort 3 (1 mg/kg), n=2 and Cohort 6 (30 mg/kg), n=5 because patients had a below limit of quantification at baseline.

FIG. 4B is a graph showing total transthyretin protein (% baseline) over time after a dose of PRX004. All patients not previously treated with patisiran (n=15). The lack of change in normal TTR levels is consistent with the proposed mechanism of action for PRX004. The data are shown with mean+Standard Error of Measurement.

FIG. 4C is a graph showing total transthyretin protein (% baseline) over time after a dose of PRX004. All patients not previously treated with patisiran (n=15). The lack of change in normal TTR levels is consistent with the proposed mechanism of action for PRX004. The data are shown with mean+Standard Deviation.

Amyloid occupancy of PRX004 was evaluated by a PK-PD model. BIACORE results show PRX004 binds with an apparent affinity (KD) of 3.2 nM (0.48 mg/ml) to aggregated TTR (affinity+avidity) and 134 nM to monomeric TTR (affinity). The higher binding strength is mainly driven by slower off-rate from amyloid versus monomers (2.0e-4s⁻¹ versus 3.7e-3s⁻¹).

Plasma Cmax is the maximum (or peak) serum concentration that a drug achieves in a specified test area after the drug is administered and before administration of a second dose. Plasma Cmax of PRX004 3 mg/kg dose at month 3 is 686 nM (103 μg/ml). Concentrations of PRX004 in heart at 3 mg/kg dose expected to be ˜68 nM (10.3 μg/ml), which would reach >90% occupancy of amyloid based on affinity measures and ex vivo data. Increase in residence time of antibody bound to amyloid due to slow off-rate might lead to accumulation and a left-shift in dose-response occupancy over time.

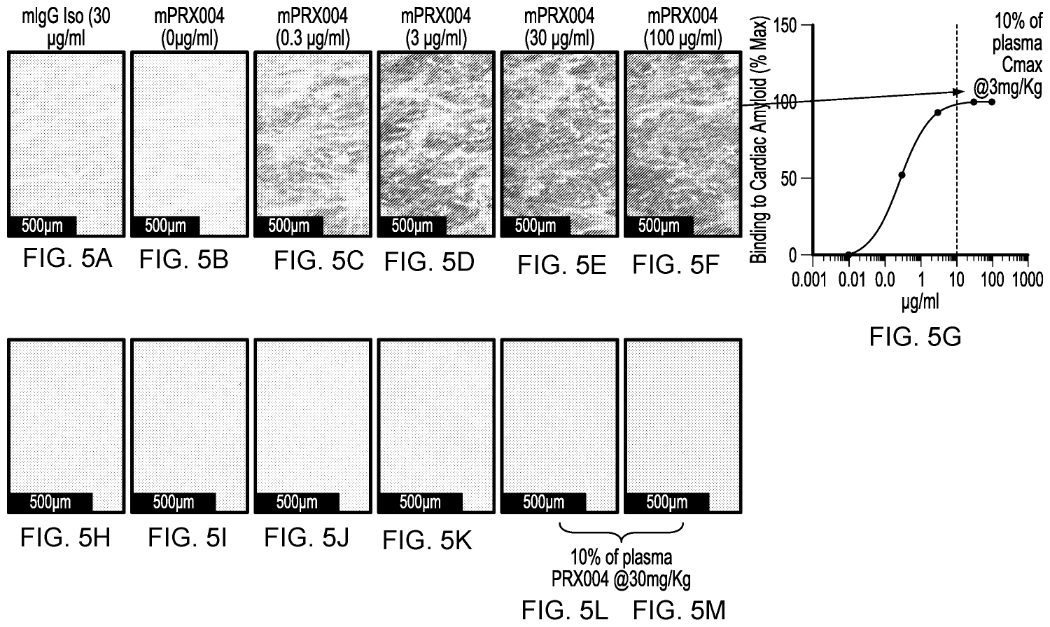

FIGS. 5A-G are images showing binding of a control antibody (FIG. 5A) and various concentrations of the murine precursor of PRX004 (mPRX004) (FIGS. 5B-F) to cardiac amyloid deposits. FIG. 5G is a graph showing a binding curve of mPRX004 to cardiac amyloid deposits. mPRX004 binds to cardiac amyloid ex vivo with the apparent affinity of 1.75 nM (0.26 mg/ml) and >90% binding expected at ˜20 nM (3 mg/ml) and above. FIGS. 5H-M are images showing binding of a control antibody (FIG. 5H) and various concentrations of the murine precursor mPRX004 (FIGS. 5I-M) in control cardiac tissue. The data show that neither the control the antibody nor mPRX004 bind the control cardiac tissue, demonstrating mPRX004's specificity for cardiac amyloid deposits.

Based on the observed PRX004 exposures; the affinity of PRX004 to amyloid, and an ex-vivo ATTR amyloid occupancy assay estimated that doses of PRX004>3 mg/kg are sufficient to opsonize and enable clearance of >90% of amyloid deposits.

Assessment of Neuropathy

Neuropathy Impairment Score (NIS) is a clinical assessment that tests muscle strength, reflex activity, and sensation of toes and fingers. For subjects with hATTR with peripheral neuropathy, neurologic function can be assessed over time using NIS. NIS involves a neurologic exam of lower limbs, upper limbs, and cranial nerves with total score of 244 (weakness [192], sensation [32], reflexes [20]) (Dyck, P. J; et al., Quantitating overall neuropathic symptoms, impairments, and outcomes. In: Dyck P J, Thomas P K, editors. Peripheral neuropathy. 4th ed. Philadelphia, PA: Elsevier Saunders; pgs. 1031-51 (2005)).

mNIS+7 is a 304-point scale that includes the same motor strength and weakness and reflex assessments. The primary difference on the mNIS+7 is the QST or quantitative sensory testing component, that replaces the sensation measure in NIS and provides a greater dynamic range, 80 points vs 32, to evidence change in the sensory component. For both scales, a higher score indicates greater impairment.

As noted above, the results of the PK-PD model above, that doses of PRX004>3 mg/kg are sufficient to opsonize and enable clearance of >90% of amyloid deposits, justified pre-specification of pooling of NIS score changes across the 3, 10 and 30 mg/kg dose cohorts.

Neuropathy severity was assessed with NIS at baseline and month 9 in 7 patients. The expected natural history of neuropathy in patients with ATTR is a progression of approximately one point per month on NIS. More specifically, over nine months one would expect NIS worsening as represented by an increase of 9.2 points. This progression is calculated based on two publications describing the change in NIS in untreated and placebo treated patients. These nine-month values are based on linear interpolation from 12-month NIS longitudinal datasets in Adams D et al., Neurology. 2015) and Berk J L et al., JAMA. 2013 (N=283, n=66).

PRX004 treated patients demonstrated a slowing of mean neuropathy progression by approximately 86% relative to what would be expected based on an analysis of published historical data in this progressive disease.

For all 7 patients, slowing of neuropathy progression was demonstrated by a mean increase from baseline in NIS of 1.29 points at 9 months. This compares favorably to the calculated mean increase in NIS of 9.2 points at 9 months in untreated and placebo-treated patients based on analysis of published historical data in patients with hereditary ATTR peripheral neuropathy. In addition to the mean improvement among the 7 patients, change in NIS for each of the 7 evaluable patients was also more favorable than published historical data.

Three patients demonstrated a numerical decrease (improvement) in NIS score from baseline (mean change of −3.33 at Month 9) and 4 had slower decline compared to the natural history (mean change +4.75 with PRX004 vs expected mean change +9 at Month 9). (See, Berk, J. L. et al., Repurposing diflunisal for familial amyloid polyneuropathy: a randomized clinical trial. JAMA, 10(24):2658-2667 (2013); Adams, D., et al. Rapid progression of familial amyloidotic polyneuropathy: a multinational natural history study. Neurology, 85(8): 675-682 (2015)). This observed decrease in NIS was encouraging in this highly progressive disease.

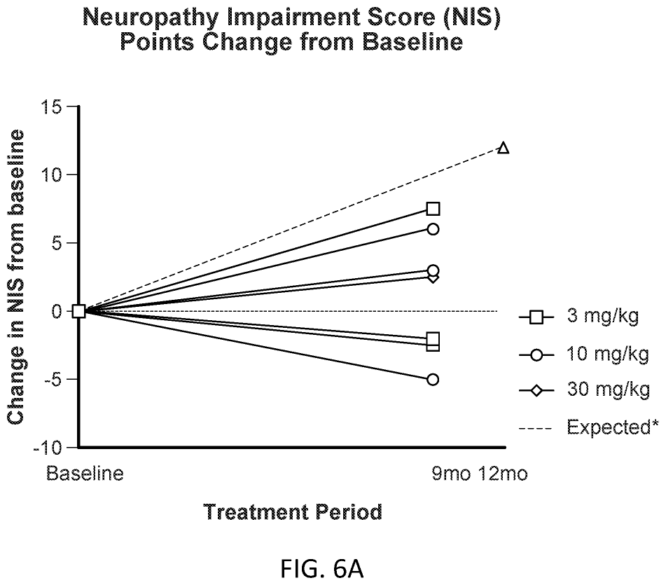

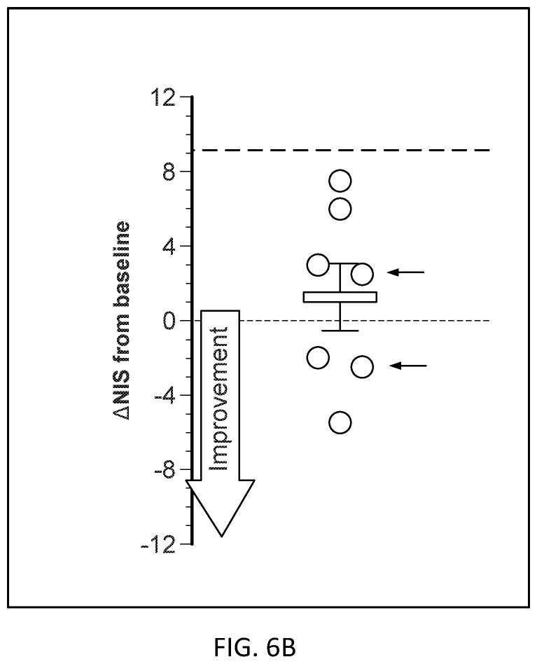

For the 2 patients who received PRX004 alone, without concomitant stabilizer therapy (dots with arrows in FIG. 6B), there was a mean change in NIS of zero at 9 months.

As a comparison, in a separate study of patisiran, a drug approved for the treatment of hereditary ATTR with peripheral neuropathy, the mean increase in NIS was 2.6 points at 18 months. See, Coelho et al. Orphanet Journal of Rare Diseases, 2020; Onpattro EPAR public assessment report EMA/55462/2018 Oct. 30, 2018.

FIG. 6A is a graph showing Changes in Neuropathy Impairment Score (NIS) from baseline over treatment interval (9 months, or 12 months) for cohorts 4 (3 mg/kg), 5 (10 mg/kg), and 6 (30 mg/kg).

FIG. 6B is another representation of the data in FIG. 5A showing Changes in NIS from baseline over treatment interval (9 months, or 12 months) for cohorts 4 (3 mg/kg), 5 (10 mg/kg) and 6 (30 mg/kg). The dots with arrows in FIG. 6B represent the data for 2 patients who received PRX004 alone, without concomitant stabilizer therapy. Table 3 summarizes the data shown in FIGS. 6A-B.

TABLE 3

| | PRX004 Phase 1 | | Natural | |
|---|---|---|---|---|
| | All Alone | PRX004 | History | Patisiran |
| ΔNIS mean | +1.29 | 0.00 | +9.2 | +2.6 |
| (n) | (n = 7) | (n = 2) | | (n = 27) |

| | | | |
|---|---|---|---|
| Time | 9 months | 9 months | 18 months |

Cardiac Systolic Function

Global longitudinal strain (GLS), which is a key measure of cardiac systolic function, was evaluated. A decrease in this measure indicates improvement.

Improvement in global longitudinal strain (GLS) was observed in all seven patients (mean change of −1.21%). For the 3 patients who had a numerical decrease in NIS score (improvement), the GLS improved by mean change of −1.51%. As with NIS, these positive results were observed in patients with or without concomitant use of stabilizer therapy. for the 2 patients who received PRX004 alone, the mean change in GLS was −2.16%.

In the APOLLO study, patisiran-treated patients had a mean increase on GLS of 0.08% at 18 months. In the placebo treated arm of APOLLO, the mean increase in GLS was 1.46% at 18 months which illustrates the normal progression in this placebo-treated patient population. See, Solomon et al. Circulation. 2019.

Together with the NIS results, it is encouraging to see improvement in cardiac systolic function in patients treated with PRX004.

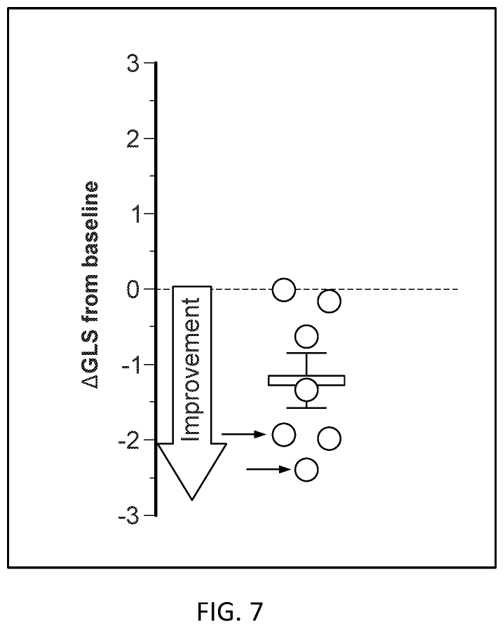

FIG. 7 is a graph showing changes in GLS from baseline over treatment (9 months, or 12 months) for cohorts 4 (3 mg/kg), 5 (10 mg/kg) and 6 (30 mg/kg). The dots with arrows in FIG. 7 represent data for patients who received PRX004 alone.

TABLE 4

| | PRX004 Phase 1 | | Placebo- | |
|---|---|---|---|---|
| | All Alone | PRX004 | treated | Patisiran |
| ΔGLS mean | −1.21% | −2.16% | +1.46% | +0.08% |
| (n) | (n = 7) | (n = 2) | (n = 36) | (n = 27) |

| | | |
|---|---|---|
| Time | 9 months | 18 months |

NYHA Classification

NHYA classification was assessed for each of the 7 patients at baseline and at 9 months. The data are summarized in Table 5 below.

TABLE 5
NYHA Classification

| Time Point | Baseline | 9 months |
|---|---|---|
| NYHA I (n) | n = 6 | n = 6* |
| NYHA II (n) | n = 1 | n = 1 |

*1 patient with NYHA I at baseline transiently moved to NYHA II at month 9, but later returned to NYHA I.

The data show that all patients were stable as their last assessed timepoint. 5 patients with NYHA I at baseline remained stable at month 9. 1 patient with NYHA at baseline transiently moved to NYHA II at month 9, but later returned to NYHA I. The 1 patient with NYHA II at baseline remained stable at month 9. Thus the data show that all 7 patients remained stable after 9 months of treatment with respect to their NYHA class at baseline.

Collectively, the data show that monthly IV infusions of PRX004 were safe and well tolerated at all dose levels up to and including 30 mg/kg, the highest dose level tested. Target engagement was demonstrated by a time and dose-dependent decrease in plasma levels of unbound misTTR. In the study we observed benefits on neuropathy and cardiac systolic function at 9 months in patients receiving PRX004. Patients showed improvement or less decline in neuropathy as measured by NIS compared to natural history. These patients all had an improvement in GLS. These observed clinical improvements suggest that amyloid-targeting provides therapeutic benefit for patients. This Phase 1 study represents the first clinical results of a therapy for the potential treatment of ATTR amyloidosis with a novel mechanism of action designed to deplete amyloid in affected organs.

### Example 2. Characterization of mPRX004

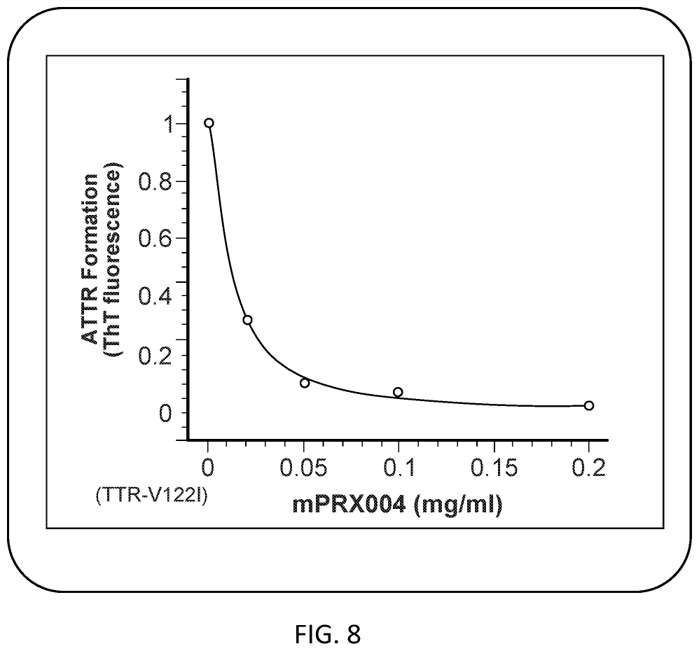

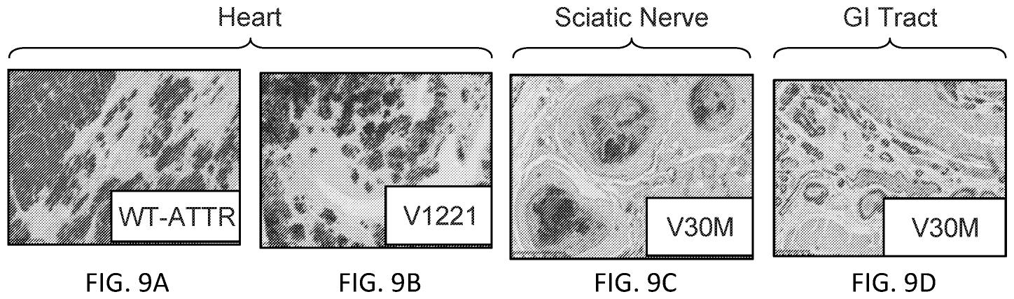

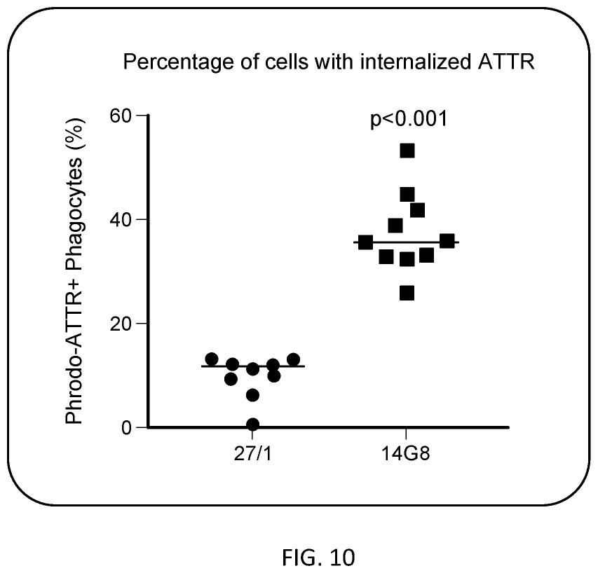

FIG. 8 is a graph showing inhibition of fibril formation in a dose dependent manner by mPRX004. FIGS. 9A-D show immunohistochemistry images of mPRX004 binding to amyloid in cardiac tissue (FIGS. 9A-B), sciatic nerve tissue (FIG. 9C), and GI tract tissue (FIG. 9D) (See, Higaki, J. N., et al., Preclinical studies of mPRX004, the murine form of PRX004, Amyloid, 23. 86-97 (2016), which is incorporated herein by reference in its entirety). FIG. 10 is a graph showing the percentage of cells with internalized (e.g.; phagocytosed) ATTR.

Collectively these data show that mPRX004 effectively inhibits fibril formation (FIG. 8), specifically binds amyloid deposits in multiple organs in both wtATTR (FIG. 9A) and hATTR subjects (FIGS. 9B-D) with specific amino acid substitutions as shown, and can clear amyloid in vivo (FIG. 10).

### Example 3. Clinical Investigation of PRX004

Further clinical investigation of PRX004 in patients with moderate-to-advanced ATTR-CM is being planned. By the time a patient is diagnosed with ATTR they may have disease classified as NYHA class III or NYHA class IV. Median overall survival is 1.3-2.1 years in NYHA class III & IV patients with ATTR. See, Pinney J et al. 2013; Gonzalez-Lopez E et al. 2017.

Tafamidis is currently the only approved therapy for patients with ATTR cardiomyopathy. However, in the ATTR-ACT phase 3 study tafamidis provided little benefit for these NYHA Class III patients and tafamidis has not been studied in patients in NYHA Class IV. See, Maurer M et al. 2018. In addition, survival benefit with tafamidis was not observed until after 18 months. Very often, moderate to advanced cardiac patients cannot wait that long. The high level of cardiac amyloid deposition in these patients leads to a severe clinical presentation and early mortality.

Silencer therapies are not approved for the treatment of ATTR cardiomyopathy. While there are clinical studies to investigate silencers for this indication, they exclude high risk patients in NYHA Class III and all patients in Class IV.

Thus, there is need to provide therapies for this moderate to advanced patient population which represents a large portion of patients with ATTR. Since PRX004 is designed to specifically target and clear amyloid from tissue it has the potential to improve survival in patients who are at high risk of early mortality due to amyloid deposition.

SEQUENCE APPENDIX

SEQ ID NO: 1 PRX004 Heavy Chain CDR1
SYTMS
SEQ ID NO: 2 PRX004 Heavy Chain CDR2
EINNSGDTTYYPDTVKG
SEQ ID NO: 3 PRX004 Heavy Chain CDR3
HYYYGGGYGGWFFDV
SEQ ID NO: 4 PRX004 Light Chain CDR1
RSSKSLLHSNGNTYLY
SEQ ID NO: 5 PRX004 Light Chain CDR2
RVSNLAS
SEQ ID NO: 6 PRX004 Light Chain CDR3
MQHLEYPLT
SEQ ID NO: 7 PRX004 Heavy Chain Variable Domain
EVQLVESGGGLVQPGGSLKLSCAASGFTFSSYTMSWVRQTPEKRLELVAEINN
SGDTTYYPDTVKGRFTISRDNAKNTLYLQMSSLKSEDTAMYYCARHYYYGG
GYGGWFFDVWGQGTLVTVSS
SEQ ID NO: 8 PRX004 Light Chain Variable Domain
DIVMTQSPLSLPVTPGEPASISCRSSKSLLHSNGNTYLYWFLQKPGQSPQLLIY
RVSNLASGVPSRFSGSGSGTDFTLKISRVEAEDVGVYYCMQHLEYPLTFGQG
TKLEIKR
SEQ ID NO: 9 PRX004 Heavy Chain Mature Amino Acid Sequence
(C-terminal lysine is optional)
EVQLVESGGG LVQPGGSLKL SCAASGFTFS SYTMSWVRQT PEKRLELVAE
INNSGDTTYY PDTVKGRFTI SRDNAKNTLY LQMSSLKSED TAMYYCARHY
YYGGGYGGWF FDVWGQGTLV TVSSASTKGP SVFPLAPSSK STSGGTAALG
CLVKDYFPEP VTVSWNSGAL TSGVHTFPAV LQSSGLYSLS SVVTVPSSSL
GTQTYICNVN HKPSNTKVDK RVEPKSCDKT HTCPPCPAPE LLGGPSVFLF
PPKPKDTLMI SRTPEVTCVV VDVSHEDPEV KFNWYVDGVE VHNAKTKPRE
EQYNSTYRVV SVLTVLHQDW LNGKEYKCKV SNKALPAPIE KTISKAKGQP
REPQVYTLPP SREEMTKNQV SLTCLVKGFY PSDIAVEWES NGQPENNYKT
TPPVLDSDGS FFLYSKLTVD KSRWQQGNVF SCSVMHEALH NHYTQKSLSL
SPGK
SEQ ID NO: 10 PRX004 Light Chain Mature Amino Acid Sequence
DIVMTQSPLS LPVTPGEPAS ISCRSSKSLL HSNGNTYLYW FLQKPGQSPQ
LLIYRVSNLA SGVPSRFSGS GSGTDFTLKI SRVEAEDVGV YYCMQHLEYP
LTFGQGTKLE IKRTVAAPSV FIFPPSDEQL KSGTASVVCL LNNFYPREAK
VQWKVDNALQ SGNSQESVTE QDSKDSTYSL SSTLTLSKAD YEKHKVYACE
VTHQGLSSPV TKSFNRGEC
SEQ ID NO: 11 Residues 89-97 of transthyretin
EHAEVVFTA

### Other Embodiments

It is to be understood that while the methods and kits have been described in conjunction with the detailed description thereof, the forgoing description is intended to illustrate and not limit the scope of the methods and kits described herein, which is defined by the scope of the appended claims. Other aspects, advantages, and modifications are within the scope of the following claims.

1. A method for of reducing the level of misfolded transthyretin (misTTR) in a human subject comprising:
administering to a human subject in need thereof an amount of an antibody or an antigen-binding fragment thereof that reduces the level of misTTR, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

2. The method of claim 1, wherein the level of misTTR in the human subject is the plasma level of misTTR in the human subject, and the administering results in a reduction in the plasma level of misTTR in the human subject of about 10% to about 99%.

3. (canceled)

4. The method of claim 2, wherein the administering results in a reduction in the plasma level of misTTR in the human subject of about 20% to about 90%, of about 40% to about 80%, or of about 50% to about 75%.

5.-32. (canceled)

33. A method of reducing the level of transthyretin amyloid deposits, transthyretin amyloid fibrils, soluble aggregate forms of misTTR or insoluble transthyretin amyloid fibrils in a human subject in need thereof comprising:
administering to a human subject in need thereof an amount of an antibody or an antigen-binding fragment thereof that reduces the level of soluble aggregate forms of misTTR, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6.

34. The method of claim 33, wherein the level of transthyretin amyloid fibrils, soluble aggregate forms of misTTR or insoluble transthyretin amyloid fibrils in the human subject is a plasma level in the human subject, wherein the administering results in an about 50% to about 99% reduction in the level of transthyretin amyloid deposits, or results in an about 50% to about 99% reduction in the plasma level of transthyretin amyloid fibrils, soluble aggregate forms of misTTR or insoluble transthyretin amyloid fibrils in the human subject.

35. (canceled)

36. The method of claim 34, wherein the administering results in an about 80% to about 99% reduction in the level of transthyretin amyloid deposits, or results in an about 80% to about 99% reduction in the plasma level of transthyretin amyloid fibrils, soluble aggregate forms of misTTR or insoluble transthyretin amyloid fibrils in the human subject.

37. The method of claim 34, wherein the administering results in an about 90% to about 99% reduction in the level of transthyretin amyloid deposits, or results in an about 90% to about 99% reduction in the plasma level of transthyretin amyloid fibrils, soluble aggregate forms of misTTR or insoluble transthyretin amyloid fibrils in the human subject.

38.-68. (canceled)

69. A method of treating ATTR amyloidosis in a human subject in need thereof comprising:
administering to a human subject in need thereof a therapeutically effective amount of an antibody or an antigen-binding fragment thereof, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6,
wherein the administering reduces the neuropathy impairment score (NIS) in the human subject in need thereof or wherein the administering reduces the global longitudinal strain (GLS) in the human subject in need thereof.

70. The method of claim 69, wherein the administering results in a reduced NIS of about 1 to about 15, or about 5 to about 15, or about 10 to about 15 in the human subject.

71.-75. (canceled)

76. The method of claim 69, wherein the administering results in a reduction in NIS of at least about 1, at least about 5, at least about 10 or at least about 20 in the human subject.

77.-86. (canceled)

87. The method of claim 69, wherein the administering results in a reduction of GLS of about 0.1% to about 10%, about 0.5% to about 10%, or about 1% to about 10% in the human subject.

88.-92. (canceled)

93. The method of claim 69, wherein the administering results in a reduction of GLS of at least about 0.5%, at least about 1.0%, at least about 2.0% or at least about 10% in the human subject.

94.-97. (canceled)

98. A method of treating heart failure in a human subject with ATTR Amyloidosis in need thereof comprising:
administering to a human subject in need thereof a therapeutically effective amount of an antibody or an antigen-binding fragment thereof, wherein the antibody or the antigen-binding fragment thereof comprises a heavy chain variable domain comprising complementarity determining regions (CDRs) of SEQ ID NO: 1, SEQ ID NO: 2, and SEQ ID NO: 3, and a light chain variable domain comprising CDRs of SEQ ID NO: 4, SEQ ID NO: 5, and SEQ ID NO: 6,
wherein the administering does not result in an increase in the New York Heart Association (NYHA) NYHA class or results in an improved NYHA class after a period of time sufficient to treat NYHA class I, II, III, or IV in the human subject.

99. (canceled)

100. The method of claim 98, wherein the ATTR Amyloidosis is hereditary ATTR Amyloidosis (hATTR).

101. The method of claim 100, wherein the hATTR is hATTR with cardiomyopathy (hATTR-CM) or hATTR with polyneuropathy (hATTR-PN).

102. The method of claim 101, wherein the ATTR Amyloidosis is wild-type amyloidosis (wtATTR).

103. The method of claim 98, wherein administering comprises administering between about 0.1 mg/kg to about 40 mg/kg of the antibody or the antigen-binding antibody fragment thereof.

104. The method of claim 103, wherein administering comprises administering between about 1 mg/kg to about 30 mg/kg of the antibody or the antigen-binding antibody fragment thereof.

105. The method of claim 104, wherein administering comprises administering between about 3 mg/kg to about 10 mg/kg or about 10 mg/kg to about 30 mg/kg of the antibody or the antigen-binding antibody fragment thereof.

106. The method of claim 103, wherein administering comprises administering about 0.1 mg/kg, about 0.3 mg/kg, about 1 mg/kg, about 3 mg/kg, about 10 mg/kg, or about 30 mg/kg of the antibody or the antigen-binding antibody fragment thereof.

107. The method of claim 103, wherein administering comprises intravenous administration.

108. The method of claim 98, wherein the administering is performed at an interval of about every 14 days, about every 2 weeks, about every 3 weeks, about every 28 days, about every 4 weeks, about monthly, about every 5 weeks, about every 6 weeks, about every 7 weeks, about every 8 weeks, or about every 2 months.

109. The method of claim 98, wherein administering is performed over a period of time of about 1 month to about 1 year.

110.-112. (canceled)


SEQUENCE LISTING (11 sequences)

SEQ ID NO: 1 - protein, 5 aa, Artificial
  PRX004 Heavy Chain CDR1
SYTMS

SEQ ID NO: 2 - protein, 17 aa, Artificial
  PRX004 Heavy Chain CDR2
EINNSGDTTYYPDTVKG

SEQ ID NO: 3 - protein, 15 aa, Artificial
  PRX004 Heavy Chain CDR3
HYYYGGGYGGWFFDV

SEQ ID NO: 4 - protein, 16 aa, Artificial
  PRX004 Light Chain CDR1
RSSKSLLHSNGNTYLY

SEQ ID NO: 5 - protein, 7 aa, Artificial
  PRX004 Light Chain CDR2
RVSNLAS

SEQ ID NO: 6 - protein, 9 aa, Artificial
  PRX004 Light Chain CDR3
MQHLEYPLT

SEQ ID NO: 7 - protein, 124 aa, Artificial
  PRX004 Heavy Chain Variable Domain
EVQLVESGGGLVQPGGSLKLSCAASGFTFSSYTMSWVRQTPEKRLELVAEINNSGDTTYYPDTVKGRFTISRDNAKNTLYLQMSSLKSEDTAMYYCARHYYYGGGYGGWFFDVWGQGTLVTVSS

SEQ ID NO: 8 - protein, 113 aa, Artificial
  PRX004 Light Chain Variable Domain
DIVMTQSPLSLPVTPGEPASISCRSSKSLLHSNGNTYLYWFLQKPGQSPQLLIYRVSNLASGVPSRFSGSGSGTDFTLKISRVEAEDVGVYYCMQHLEYPLTFGQGTKLEIKR

SEQ ID NO: 9 - protein, 454 aa, Artificial
  PRX004 Heavy Chain Mature Amino Acid Sequence
EVQLVESGGGLVQPGGSLKLSCAASGFTFSSYTMSWVRQTPEKRLELVAEINNSGDTTYYPDTVKGRFTISRDNAKNTLYLQMSSLKSEDTAMYYCARHYYYGGGYGGWFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Artificial
  PRX004 Light Chain Mature Amino Acid Sequence
DIVMTQSPLSLPVTPGEPASISCRSSKSLLHSNGNTYLYWFLQKPGQSPQLLIYRVSNLASGVPSRFSGSGSGTDFTLKISRVEAEDVGVYYCMQHLEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 9 aa, Artificial
  Residues 89-97 of transthyretin
EHAEVVFTA